(19) United States
(12) Patent Application Publication
(10) Pub. No.: US 2011/0244487 A1
(43) Pub. Date: Oct. 6, 2011

(54) METHODS FOR TESTING BINDING OF A LIGAND TO A G PROTEIN-COUPLED RECEPTOR

(75) Inventors: Jeff Horton, Cardiff (GB); Peter Tatnell, Cardiff (GB)

(73) Assignee: GE HEALTHCARE UK LIMITED, LITTLE CHALFONT (GB)

(21) Appl. No.: 13/132,642

(22) PCT Filed: Dec. 4, 2009
(86) PCT No.: PCT/EP09/66418

§ 371 Date: Jun. 3, 2011

(30) Foreign Application Priority Data
Dec. 5, 2008 (GB) 0822259.8

(51) Int. Cl. G01N 33/566; C12N 5/10

(52) U.S. Cl. 435/7.21; 435/7.8; 435/325

(57) ABSTRACT

The present invention relates to methods for testing for the binding of a ligand to a G Protein-Coupled Receptor. In particular, the methods of the invention are useful in high throughput screening for ligands which bind to G Protein-Coupled Receptors.

### CROSS-REFERENCE TO RELATED APPLICATIONS

This application is a filing under 35 U.S.C. §371 and claims priority to international patent application number PCT/EP2009/066418 filed Dec. 4, 2009, published on Jun. 10, 2010 as WO 2010/063832, which claims priority to application number 0822259.8 filed in Great Britain on Dec. 5, 2008.

### FIELD OF THE INVENTION

The present invention relates to the field of cell biology, molecular biology and drug screening. In particular, the invention relates to G Protein-Coupled Receptors (GPCRs) and to methods for testing the binding of ligands to GPCRs.

### BACKGROUND OF THE INVENTION

### G Protein-Coupled Receptors

G protein-coupled receptors (GPCRs), also known as seven transmembrane domain receptors, 7TM receptors and G protein-linked receptors (GPLR), comprise a large protein family of trans-membrane receptors that bind molecules outside the cell and activate signal transduction pathways and, ultimately, cellular responses. GPCRs are found only in eukaryotes, including yeast, plants, flagellate protozoa, animals. The ligands that bind and activate these receptors include light-sensitive compounds, odours, pheromones, hormones, neurotransmitters, and drugs, and vary in size from small molecules such as peptides to large proteins. As GPCRs are involved in many diseases, they are the target of many modern medicines. Drugs active at GPCRs have therapeutic benefit across a broad spectrum of human diseases as diverse as pain, cognitive dysfunction, hypertension, peptic ulcers, rhinitis, asthma, inflammation, obesity and cancer, as well as cardiovascular, metabolic, gastrointestinal, visual and neurodegenerative diseases. Of the total clinically marketed drugs, greater than 30% are modulators of GPCR function, representing 9% of global pharmaceutical sales, making GPCRs the most successful of any target class in terms of drug discovery. GPCRs represent the single most important class of drug targets and significant targets in drug discovery. Indeed, 20% of the top fifty best selling drugs act at GPCRs, which equates to approximately $25 billion in terms of pharmaceutical sales per annum. However, current drugs exhibit their activity at less than 10% of known GPCRs, implying that there is large potential for further discovery. Indeed, the DNA sequences of a large number of GPCRs can be found in public databases, among other sources, leading to identification of putative so-called “orphan receptors”. These orphan receptors are defined as those not acted upon by a known endogenous ligand. One challenge for the drug development industry is to associate the many orphan GPCRs with disease to potentially identify novel pharmaceutical agents of the future.

GPCRs are closely associated with heterotrimeric G-proteins that are bound to the inner face of the plasma membrane. G-proteins are key molecular components in the intracellular signal transduction following ligand binding to the extracellular domain of a GPCR. The G-protein subunits historically are designated α, β, and γ, and their classification is largely based on the identity of their distinct α subunits, and the nature of the subsequent transduction event (Table 1). Further classification of G-proteins has come from cDNA sequence homology analysis. G-proteins bind either guanosine diphosphate (GDP) or guanosine triphosphate (GTP), and possess highly homologous guanine nucleotide binding domains and distinct domains for interactions with receptors and effectors.

When the GPCR “system” is inactive (i.e. in the absence of ligand), GDP is bound to the Gα subunit. An agonist-receptor complex induces conformational changes in the GPCR/G-protein complex, which facilitates preferential binding of GTP to the Gα subunit, in part by promoting the dissociation of bound GDP. This so-called “guanyl nucleotide exchange” is critical. Binding of GTP activates the Gα subunit, leading to dissociation through space from the Gβγ dimer. The Gα and Gβγ subunits are then able to subsequently activate, either independently or in parallel, downstream effectors such as adenylate cylase, calcium, phospholipase activity or other ions.

Termination of signal transduction results from hydrolysis of bound GTP to GDP by a GTPase enzyme that is intrinsic to the α subunit, leading to re-association of α and βγ subunits. Thus, G-proteins serve as regulated molecular switches capable of eliciting bifurcating signals through α and βγ subunit effects. The switch is turned on by the receptor and it turns itself off within a few seconds, a time sufficient for considerable amplification of signal transduction.

TABLE 1
Examples of the relationship of G Protein-Coupled
Receptors and Signalling Pathways

| G- Protein | |
|---|---|
| Subunit Regulation | Effectors/Signalling Pathways |
| αs | Adenylate cyclase (cAMP) ⇑ |
| αi | Adenylate cyclase (cAMP) ↓ |
| αo | Ca²⁺ ↓ |
| αq | Phospholipase C (IP₃) ⇑ |
| α13 | Na⁺/H⁺ exchange ⇑ |
| αt | cGMP-phosphodiesterase (vision) ⇑ |
| αolf | Adenylate cyclase (cAMP) ⇑ |
| βγ | K⁺ channels |
| βγ | Adenylate cyclase (cAMP) ⇑ or ↓ |
| βγ | Phospholipase C (IP₃) |

### Structure of GPCRs

GPCRs are integral hydrophobic membrane proteins that span the plasma membrane in seven α-helical segments. The extracellular binding site for small GPCR-active ligands is a pocket within the bundle of membrane-spanning helices, but a substantial extracellular domain is important for the binding of the negatively charged ligands. GPCRs are activated by an external signal in the form of a ligand or other signal mediator. This creates a conformational change in the receptor, causing activation of a G-protein. Further effect depends on the type of G-protein. The receptors interact with G proteins at their cytoplasmic face, and it has been possible to define specific regions within GPCR structures that are responsible for regulation of and selectivity among the different G-proteins.

In order to study GPCR activation, a variety of functional biochemical and cellular assay methodologies are typically used. Examples of functional assay systems for monitoring GPCR activation include the intracellular measurement of the GPCR effector targets, cAMP, cGMP and IP₃. A number of homogeneous assay methodologies such as Scintillation Proximity Assay (SPA), Fluorescence Polarization (FP) and Enzyme Fragment Complementation (EFC) have been successfully used for the measurement of these agents. Furthermore, as described above, ligand-induced stimulation of GPCRs results in the exchange of GDP for GTP, and this event can be monitored by the binding of radiolabelled [³⁵S] GTPγS, for example.

In the process of drug discovery and lead optimisation, there is a requirement for faster, more effective, less expensive and especially information-rich screening assays that provide simultaneous information on various compound characteristics and their affects on various cellular pathways (i.e. efficacy, specificity, toxicity and drug metabolism). Such assays would allow drug discovery to identify drug candidates capable of activating or blocking GPCR signalling. Thus, for drug discovery, there is a need to quickly and inexpensively screen large numbers of chemical compounds to identify new drug candidates, including receptor agonists, inverse agonists and antagonists as well as inhibitors of GPCRs and GPCR-dependent pathways. These chemical compounds may be collected in large libraries, sometimes exceeding one million distinct compounds.

Traditional biochemical approaches for assaying GPCRs have relied upon measurements of ligand binding, for example with filter binding assays (heterogeneous) or with SPA (homogeneous). Although such assays are inexpensive to carry out, development time can be lengthy in some cases. A major problem is that the development of a traditional assay requires specific reagents for every target of interest including purified protein for the target against which the screen is to be run. Often it is difficult to express the protein of interest and/or to obtain a sufficient quantity of the protein in pure form.

Although binding assays are the gold standard for pharmacology and studies of structure-activity relationships (SAR), it is not usually possible to perform target validation with binding assays. The increased numbers of drug targets identified by genomic approaches has driven the development of gene-to-screen approaches to interrogate poorly-defined targets, many of which rely on cellular assay systems. Speculative targets are most easily screened in a format in which the target is expressed and regulated in the biological context of a cell, in which all of the necessary components are pre-assembled and regulated. Cell-based assays are also critical for assessing the mechanism of action of new biological targets and biological activity of chemical compounds. In particular, there is a need to “de-orphanise” those GPCRs for which natural activating ligand has not been identified. Various approaches to “de-orphanisation” have been adopted including array-screening against families of known ligands. Current cell-based assays for GPCRs include measures of pathway activation (Ca²⁺ release, cAMP generation or transcriptional activity); measurements of protein trafficking by tagging GPCRs and down stream elements with GFP; and direct measures of interactions between proteins using Förster resonance energy transfer (FRET), bioluminescence resonance energy transfer (BRET) or yeast two-hybrid approaches.

### Definitions

The meaning of the terms “agonist”, “inverse agonist” and “antagonist” as used herein are based on the definition given in: “Definitions, GlaxoWellcome Pharmacology Guide”, which can be found online.

An “agonist” is a compound or drug which binds to a receptor and activates it, producing a pharmacological response (e.g. contraction, relaxation, secretion, enzyme activation, etc.).

An “inverse agonist” is a compound or drug which produces an effect opposite to that of an agonist, yet acts at the same receptor. The best established examples act at the benzodiazepine receptor. Such compounds have also been described as “negative antagonists”, or as having “negative efficacy”.

An “antagonist” is a compound or drug which attenuates the effect of an agonist. It may be competitive, i.e. it binds reversibly to a region of the receptor in common with an agonist but occupies the site without activating the effector mechanism. The effects of a competitive antagonist may be overcome by increasing the concentration of agonist, thereby shifting the equilibrium and increasing the proportion of receptors which the agonist occupies.

Alternatively, antagonists may be non-competitive, where no amount of agonist can completely overcome the inhibition once it has been established. Non-competitive antagonists may bind covalently to the agonist binding site (“competitive irreversible antagonists”), in which case there is a period before the covalent bond forms during which competing ligands can prevent the inhibition. Other types of non-competitive antagonists act allosterically at a different site on the receptor or an associated ion channel.

The term “testing” as used herein, shall include but not be limited to, detecting, measuring and/or quantifying.

“Binding” is defined herein as an event which involves an agent or molecule selectively interacting with one or more sites on another molecule.

A “ligand” as used herein, shall mean a substance or compound that is able to bind to form a complex with a biomolecule to serve a biological purpose such as triggering a biological response.

An “homogeneous assay is defined herein as a method that does not involve a wash step, for example a step without physical separation.

“Optical signal”, as used herein, shall mean light emission of any wavelength. For the avoidance of doubt, this includes luminescence, fluorescence and any form of electro magnetic radiation such as x-rays.

“Fluid sample” shall mean a liquid solution or a suspension.

### Cell Based Assays

Traditional cell-based assays for GPCRs often rely upon measurements of intracellular calcium flux. Calcium release from intracellular stores is stimulated by specific classes of GPCRs upon their activation; in particular those GPCRs that couple to a G-protein known as Gq (or Gq/11). Fluorescent and luminescent assays of calcium release have been generated by loading cells with dyes that act as calcium indicators. Fluorescent calcium indicators such as Fura-2, Indole-1, Fluo-3 and calcium green have been widely used for measurement of intracellular calcium measurement. Such indicators and associated instrumentation such as FLIPR (Molecular Devices, Sunnyvale, Calif., USA) are well established tools. Luminescent assays of calcium transients can be also carried out, by the introduction of aequorin into cells, usually with genetic engineering techniques. Aequorin emits blue light in the presence of calcium, and the rate of photon emission is proportional to the free calcium concentration within a specific range. Cells expressing the GPCR of interest are loaded first with coelenterazine to activate the aequorin, and then the compounds to be tested are added to the cells and the results measured with a luminometer. To extend these assays to non-Gq-coupled receptors, various engineering strategies have been used, including the use of a promiscuous Gα protein construct such as Gα16 that is capable of coupling a wide range of GPCRs to phospholipase C (PLC) activity and calcium mobilisation.

Adenylyl cyclases are a family of membrane-bound enzymes that are linked to G protein-coupled receptors and influence the regulation of cell function in virtually all cells. cAMP is synthesized by adenylyl cyclase in response to activation of many receptors; stimulation is mediated by Gs, and inhibition by one or more closely related G proteins termed Gi's. There exist at least ten tissue-specific adenylyl cyclases each with its unique pattern of regulatory responses. Several adenylyl cyclase isozymes are inhibited by the G protein βγ subunits, which allow activation of G proteins other than Gs to inhibit cyclase activity. Other isozymes are stimulated by Gβγ subunits, but this stimulation is dependent upon concurrent stimulation by the α subunit of Gs. Still other isozymes are stimulated by Ca²⁺ or Ca²⁺⁻ calmodulin complexes. Finally, each of the isozymes has its own pattern of enhancement or attenuation by phosphorylation or other regulatory influences, providing a broad array of regulatory features to the target cells where these isoforms are expressed.

cAMP is a ubiquitous second messenger and functions as one of many signalling molecules enabling cells to respond to external signals. cAMP assays are used to monitor cellular responses to either Gs or Gi-coupled receptor activation. Typically, with cAMP, the binding of a hormone, agonist or neuromodulator to its receptor is followed by activation or inhibition of a G-protein which, in turn, activates adenylate cyclase, evoking the generation of cAMP from ATP. The activation of protein kinase A by cAMP results in the phosphorylation of specific substrates, which include enzymes, ion channels and transcription factors. Because cAMP can activate a cascade of reactions, the involvement of cAMP greatly amplifies the cellular response to a variety of drugs and hormonal stimuli. Therefore, measurement of intracellular cAMP generation has become an established means of screening for antagonists and agonists of receptors linked to adenylate cyclase via either inhibitory or stimulatory G-proteins.

Fluorescent dyes, and fluorescent proteins such as GFP, YFP, BFP and CFP have also been used as cellular sensors of cAMP and calcium. The first protein indicator for cAMP consisted of the cyclic AMP-dependent protein kinase, PKA, in which the catalytic and regulatory subunits were labelled with fluorescein and rhodamine, respectively, so that cAMP-induced dissociation of the subunits disrupted FRET between the dyes. Replacement of the dyes by GFP and BFP made this system genetically encodable and eliminated the need for in vitro dye conjugation and microinjection.

Transcriptional reporter assays provide a measurement of pathway activation or inhibition in response to an agonist/antagonist, and have been used extensively in GPCR studies. Reporter-gene assays couple the biological activity of a receptor to the expression of a readily detectable enzyme or protein reporter. Synthetic repeats of a particular response element can be inserted upstream of the reporter gene to regulate its expression in response to signalling molecules generated by activation of a specific pathway in a live cell. Such drug screening systems have been developed with a variety of enzymatic and fluorescent reporters, including β-galactosidase, luciferase, alkaline phosphatase, green fluorescent protein (GFP), β-lactamase) and others. Transcription reporter assays are highly sensitive screening tools; however, they do not provide information on the mechanism of action of the compound. The latter would enable mapping of the components of the pathway, leading to transcription, or enable studies of the individual steps within signalling cascades.

High content screening (HCS) is an approach that, in one format, relies upon imaging of cells to detect the sub-cellular location and trafficking of proteins in response to stimuli or inhibitors of cellular processes.

Kimple et al. (2006) Combinatorial Chemistry & High Throughput Screening describes established and emerging fluorescence—based assays for G-protein function.

Fluorescent probes can be used in HCS. For example, GTP has been labelled with the fluorescent dye, BODIPY®, and used to study the on/off-rates of GTP hydrolysis by G-proteins.

Fluorescein-labelled myristoylated Gαi has also been used as a ligand that binds Gβγ in order to study the association and dissociation of G-protein subunits.

GFP has been used to analyse key signalling events within cells. By fusing in-frame a cDNA for GFP to a cDNA coding for a protein of interest, it is possible to examine the function and fate of the resulting chimera in living cells. This strategy has now been applied to nearly all known elements of G-protein coupled pathways including the receptors themselves, G-protein subunits such as Gα; β-arrestin, RGS proteins, protein kinase C and other intracellular components of G-protein-coupled pathways. For example, GPCRs have been tagged with GFP in order to monitor receptor internalization. A fusion protein comprising GFP-β-arrestin has been shown to co-localise with thyrotropin-releasing hormone receptor 1 in response to agonist. GFP has been introduced internally to G-proteins, creating a Gα/GFP chimera, which has been shown to translocate to the cell membrane upon GPCR activation. GFP tagging has also been used to monitor intracellular signalling events. GFP tagged RGS proteins were selectively recruited to the plasma membrane by G-proteins and their receptors. GFP-tagged protein kinase C (PKC), which is activated by the release of diacylglycerol from cell membranes, has been used to monitor translocation of the kinase in response to cell signalling.

In addition, GFP-tagged connexion has been used to monitor intracellular calcium flux.

U.S. Pat. No. 5,891,646 and U.S. Pat. No. 6,110,693 (Norak Bioscience) describe GFP-tagged β-arrestin and this has been used to monitor GPCR activation by imaging the subcellular redistribution of β-arrestin in response to agonist. However, the signal generated from GFP translocation methods is very low and is prone to instrument interference which can result in poor assay results

WO 2005/121755 and Leifert et al., (2006) Anal. Biochem., 355, 201-212 describe a cell-free GPCR and ligand assay using chemically-generated fluor molecules in a FRET assay.

An homogeneous GTP binding assay for G protein-coupled receptors based on time resolved FRET has previously been described (Frang, H., et al., GTP binding assay for GPCRs based on TR-FRET, Poster PO 8123, Ninth Annual Society for Biomolecular Screening, Portland, Oreg., 21-25 Sep. 2003). In this assay, a biotinylated BIOKEY® peptide is employed that recognizes only the GTP bound form of the Gα subunit. The biotinylated peptide enables binding of streptavidin-europium in close proximity to an acceptor-labelled GTP, which is also bound to the Gα subunit. FRET occurs as a result of interaction between the streptavidin-europium (donor) and the fluorescently-labelled GTP analogue (Alexa647-GTP).

WO 2006/035207 (GE Healthcare UK Limited) describes fluorescent cyanine dye labeled nucleotide analogues in which the cyanine dye is coupled to the γ-phosphate group of a nucleoside triphosphate. These GTPase resistant analogues can be used in an homogenous FRET-based assay to measure the binding of guanine nucleotides to GPCR polypeptides, or alternatively, to measure the effect of an exogenous ligand on GPCR binding. Further uses of similar GTPase resistant GTP analogues in GPCR binding assays are disclosed in WO2006/035208 (GE Healthcare UK Limited).

While a variety of fluorescent dye-nucleotide conjugates are available, the selection of a particular fluorescent label for use in a protein binding assay can be problematic, since the electronic and spatial requirements of the binding site of the protein of interest are difficult to predict a priori. There is therefore, still a requirement for new GTP analogues that may be used for quantitating G-proteins and for studying the kinetics of agonist induced guanine nucleotide exchange in in vitro assays and in cellular systems.

### Enzyme Complementation

β-galactosidase (β-gal) is a tetrameric enzyme with a MW of 464,000. Each identical subunit contains 1021 amino acids, encoded in E. coli by the lacz gene of the lac operon promoter. In E. coli, intracistronic β galactosidase (β-gal) complementation is a naturally occurring phenomenon, and involves α and ω complementation of amino and carboxyl-terminal domains of the lac Z enzyme.

Ullmann et al. (Ullmann A, Perrin D, Jacob F, Monod J (1965). J Mol Biol., 12, 918-923) described the complementation of β-gal in E. coli. A peptide was found (Peptide “ω”) that was present in extracts of various mutants (“ω donors”) of the lacz gene. The ω peptide complemented β-gal activity when added to extracts containing a β-gal (-ve) mutant (“ω acceptor”). The ω enzyme acceptor peptide (EA) has since been found to lack residues 11-41, and is frequently referred to as the M15 protein, since it is a product of the lac Z M15 allele. Sucrose density assessments suggested a MW of 30,000 to 40,000 for the ω peptide and in an operator-distal segment of the z gene. A following publication by Ullmann et al. (Ullmann A, Jacob F, Monod J, 1967 J Mol Biol., 24, 339-343) described how extracts from various β-gal-negative mutants were screened for their capacity to complement with extracts of partial deletions of the operator-proximal segment (“α”) of the z gene. Together, the operator-distal (ω) and the operator-proximal (α) part of the z gene account for about one-half of both the structural length and MW of the lacZ gene for β-gal.

Zamenhoff and Villarejo (Zamenhof P, Villarejo M, 1972, J Bacteriol., 110, 171-178) demonstrated in vivo α-complementation of β-gal in 16 lacZ gene terminator (nonsense) mutant strains of E. coli upon introduction of a gene fragment specifying production of a mutant lacZ polypeptide containing a small deletion in the N-terminal region of the enzyme monomer.

Since then, many sequence variants of donor and acceptor species of β-gal have been described, reviewed by Eglen (Eglen R, 2002, Assay and Drug Development Technologies, 1, 97-105; DiscoveRx). In particular, a variation developed by DiscoveRx is a system for complementation of a small 4 kDa α fragment donor (ED) peptide (termed “ProLabel”) with an ω deletion mutant of the enzyme acceptor (EA).

Further work reviewed by Olson and Eglen (Olson K & Eglen R, 2007, Assay and Drug Development Technologies, 5, 137-144) describes the 47-mer enzyme donor (ED) sequence.

Henderson et al., (1986) Clinical Chemistry, 32(9), 1637-1641 (Microgenics) describes genetic engineering of β-galactosidase which led to the development of a homogeneous immunoassay system.

U.S. Pat. No. 5,120,653 (Microgenics) describes a vector comprising a DNA sequence coding for an enzyme-donor polypeptide.

U.S. Pat. No. 5,643,734 and U.S. Pat. No. 5,604,091 (Microgenics) describe methods and compositions for enzyme complementation assays for qualitative and quantitative measurement of an analyte in a sample.

WO 2008/085481(Leland Stanford Junior University) describes the detection of sub-cellular compartment localization of a molecule using a reduced affinity enzyme complementation reporter system of β-galactosidase. Methods for detecting translocation of a cell-surface receptor to a sub-cellular compartment, such as an endosome are disclosed.

WO2001/58923 (Tropix) describes a method to interrogate G-protein coupled receptor function and pathways using an arrestin together with a particular mutant enzyme-fragment complementation system.

Weber et al., (2004) Assay and Drug Development Technol., 2, 39-49) reports the use of an enzyme fragment complementation (β-galactosidase) based cAMP assay as functional screens for GPCRs.

Zhao et al., (2008) J. Biomolecular Screening, 13, 737-747 describes a homogeneous, functional assay system that directly monitors G-protein coupled receptor activation using enzyme fragmentation complementation of β-galactosidase and the translocation of β-arrestin.

Yan et al., (2002) J. Biomolecular Screening, 7, 451-459 discloses a cell-based high-throughput screening assay system for monitoring G-protein coupled receptor activation using β-galactosidase enzyme fragment complementation. The method is based on the interaction between β-arrestin and uses a pair of inactive β-galactosidase deletion mutants as fusion partners to the protein targets of interest. To monitor GPCR activation, stable cell lines expressing both GPCR and β-arrestin-β-galactosidase fusion proteins were generated.

Carter & Hill (2005) J. Pharmacology & Exp. Therapeutics, 315, 839-848 describes the use of an antibody-based assay for GPCRs using cAMP as target with enzyme fragment complementation of β-galactosidase. In addition, this paper describes C2C12 cells stably transfected with β-adrenoreceptor-β-gal and β-arrestin fusion proteins of β-galactosidase, and, enzyme fragment complementation following cell stimulation with isoprenaline and salmeterol.

WO 2003/021265 (DiscoveRx) describes a genetic construct intracellular monitoring system provided for producing biologically active fusion proteins comprising a sequence encoding an enzyme donor (“ED”) sequence of fused in reading frame to a sequence encoding a surrogate of a mammalian protein of interest, where the fusion protein has the function of the natural protein. Furthermore, a vector is described comprising a transcriptional and translational regulatory region functional in a mammalian host cell, a sequence encoding the ED joined to a multiple cloning site, an enzyme acceptor (“EA”) protein or enzyme acceptor sequence encoding such protein that is complemented by the ED to form a functional enzyme such as β-galactosidase. Mammalian cells are employed that are modified to provide specific functions.

U.S. Pat. No. 7,135,325 (DiscoveRx) describes short enzyme donor fragments of β-galactosidase of not more than 40 amino acids.

WO 2006/004936 (DiscoveRx) describes methods for determining the intracellular state of a protein as well as modifications to the protein. The method involves introducing a surrogate fusion protein comprising a member of an enzyme fragment complementation complex and a target protein. After exposing cells transformed with the surrogate fusion protein to a change in environment (e.g. a candidate drug), the cells are lysed, the lysate separated into fractions or bands by gel electrophoresis and the proteins transferred by Western blot to a membrane. The bands on the membrane are developed using the other member of the enzyme fragment complementation complex and a substrate providing a detectable signal.

US2007/0105160 (DiscoveRx) describes methods and compositions for determining intracellular translocation of proteins employing β-galactosidase fragments that independently complex to form an active enzyme. Engineered cells have two fusion constructs: one fragment bound to a protein of interest; and the other fragment bound to a compartment localizing signal. The cells are used to screen compounds for their effect on translocation, where a substrate containing high ionic strength solution is used for detection of the enzyme complex.

WO2005/047305 (DiscoveRx) describes methods and compositions for the determination of populations of proteins and receptors at cellular membranes. The methods involve the use of a transformed or transfected cells having a genetic capability to express a fusion protein comprising a cellular membrane protein fused to a signal producing polypeptide through a proteolytic susceptible sequence. In one example, the signal producing peptide is referred to as the enzyme donor. The signal producing peptide is one of a pair of fragments of an enzyme that is reconstituted when the two fragments, the enzyme donor and the enzyme acceptor, complex together. An example of such an enzyme is β-galactosidase. Specific target groups of proteins are included such as G-protein coupled receptors.

In addition to enzyme complementation of β-Gal, complementation is a common phenomenon now reported for other proteins, including dihydrofolate reductase (Remy I & Michnick S, 2001, Proc Natl Acad Sci USA., 98, 7678-7683), β-lactamase (Wehrman T et al., 2002. Proc Natl Acad Sci USA, 99, 3469-3474), luciferase (Ozawa T., et al., 2001. Anal Chem., 73, 2516-2521), ubiquitinase (Rojo-Niersbach E et al., 2000, Biochem J., 348, 585-590), alkaline phosphatase (Garen A., & Garen S., 1963, J. Mol. Biol. 7, 13-22) and tryptophan synthase (Yanofsky, C., & Crawford, I. P., 1972, Enzymes 7, 1-31).

### G-Protein Coupled Receptor Kinases (GRK)

Membrane associated heterotrimeric G-proteins bind to cell-surface GPCRs and are integral in the transmission of signals from outside the cell. The Gβγ homodimer binds tightly to the GDP-bound Gα subunit, enhancing Gα coupling to the receptor and inhibiting the release of GDP. Agonist binding to the GPCR promotes the replacement of GDP for GTP on the Gα subunit this, changes the conformation facilitating the dissociation of Gβγ.

GTP-bound Gα and free Gβγ both initiate signals by interactions with downstream effector proteins. On uncoupling from the G-proteins the GPCRs becomes phosphorylated by G-protein coupled receptor kinases (GRK) which reside in the cytosol and translocate to the plasma membrane on GPCR activation where they anchor to the free Gβγ subunit. Members of the arrestins family of proteins then bind to the phospohorylated receptor terminating signal transmission and initiating receptor internalization. The intrinsic Gα GTPase activity returns the protein to the GDP-bound state when the heterotrimeric G-protein complex reforms. Re-association of the complex obscures critical effector contact sites on both Gα and Gβγ, terminating all effector interactions and thereby all downstream signalling events (Siderovski et al., 2005 Int. J. Biol Sci. 1, 51-66).

At present 16 genes are known to encode for Human Gα subunits, 5 for Gβ and 12 genes for Gγ. Classically, G proteins are divided into four families based upon their Gα subunits—Gαi, Gαs, Gαq/11 and Gα12/13.

### Tagging the N-Terminus of Gβ and Gγ Subunits

Both the Gβ and Gγ subunits have been N-terminally tagged with Enhanced Cyan Fluorescent Protein, ECFP (Bunnemann et al., 2003, PNAS 100 16077-16082). In these experiments the authors demonstrated a functional signaling pathway from receptors to heterotrimeric G proteins to downstream signaling events. The ECFP-tagged proteins were functionally equivalent to wild-type proteins.

This study demonstrates that the N-terminals of both Gβ and Gγ subunits can be fused to reporter molecules (e.g. ECFP or β-Gal donor peptide) without affecting heterotrimeric G-protein function. This is depicted in FIGS. 1 and 2, which illustrates the site of attachment of the reporter molecule at the N-terminus of the Gβ and Gγ subunits, respectively.

### GRK2 Interaction with the Gβ Sub-Unit

The GRKs consist of a family of six related iso-enzymes (GRKs1-6) that transfer phosphate groups onto serine and threonine residues located close to the C terminal of GPCRs. GRK2 consists of 3 domains, an N-terminal RGS (regulator of G-protein signaling), a central protein kinase domain and a C-terminal pleckstrin homology (PH) domain. The RGS and kinase domains are common to all GRKs whereas the PH domain is unique to GRK2 and GRK3. On GPCR activation GRK2 binds the Gβγ subunit and the process of desensitization begins by GRK phosphorylating the GPCRs. GRK2 is ubiquitously expressed and can phosphorylate many different GPCRs. The crystal structure of the GRK2 and G₁βγ₂ complex has been solved and reveals how the RGS, kinase and PH domains integrate their activities to bring the enzyme to the membrane in an orientation that facilitates GPCR phosphorylation. The GRK2 PH domain binds exclusively to the Gβ subunit. The footprint of the PH domain on the Gβγ subunit overlaps extensively with the binding site for Gα and other Gβγ effectors. Therefore GRK2 also inhibits G-protein signaling by blocking the interactions of Gα and Gβγ subunits preventing re-association. Four regions within the PH domain contribute to the Gβ interaction. These form a continuous surface and include strands β1 to β4 and a portion of the C-terminal helix. The Gβγ propeller region responsible for binding the PH domain (and Gα) is extremely acidic, accordingly the GRK2 PH domain possesses many basic residues.

The GRK2 PH domains binds exclusively to the Gβ protein. This is shown schematically in FIG. 3. The structure indicates that the C-terminal end of the PH domain contributes to Gβ binding. These are positioned 22 residues upstream from the extreme C-terminus of GRK2. These 22 residues exist in a random flexible coil and therefore are difficult to visualize in crystals. However, they are well suited as a flexible linker region connecting the β-gal acceptor peptide to GRK2. Note in this format that the acceptor peptide C-terminal region is able to form an inter-molecular dimer with other β-Gal acceptor peptides.

The N-terminal regions of both the Gβ and Gγ are not involved in the PH domain interaction thereby allowing their tagging with EGFP or the β-gal donor peptide.

At present no structure has been determined for the related GRK3 interaction. However, on GPCR activation GRK3 translocates to the plasma membrane from the cytoplasm. GRK3 also possesses a C-terminal PH domain that over its 111 residues contains 13 conservative and 14 non-conservative amino acid differences from that of GRK2. Some of these changes are in areas known to be involved in the GRK2 Gβ interaction. These changes imply that GRK3 may possess a different G-protein binding preference. It has been demonstrated that the over-expression of GRK3 is accompanied by the agonist-mediated phosphorylation of the Gα_{q/11}-linked mACh receptor

The beta galactosidase crystal structure explains a-complementation. The N-terminal donor residues (˜50) are positioned on the surface of the protein. Amino acid residues 13 and 15 contribute to the activating interface and residues 29-33 pass through a “tunnel” formed by an intra-molecular domain-domain interaction. Essentially, the donor peptide threads through this tunnel and restores the interactions present in the wild-type enzyme generating enzyme activity. Beta galactosidase a-complementation forms an enzyme with catalytic and substrate affinities equivalent to those of the wild-type enzyme (Olson et al., 2007 Assay & Drug Dev Tech 5, 137-144).

To generate enzyme activity a free β-Gal N-terminus is optimally required. Therefore this essentially controls the choice of which β-Gal peptide fragment is coupled to which of the protein partners. A free N-terminal peptide donor fragment can only be accommodated by coupling to the N-terminals of the Gβ and Gγ proteins (see FIGS. 1 and 2).

Table 2 gives examples of some commercially vectors.

TABLE 2
cDNAs available from Mammalian Gene
Collection (MGC), NIH, Maryland, USA

| cDNA | Species | MGC No. | Image clone No. | Accession No. |
|---|---|---|---|---|
| GRK2 | Human | MGC46122 | 5585846 | BC037963 |
| (ADRBK1) | | | | |
| GRK3 | Human | MGC46121 | 5590378 | BC036797 |
| (ADRBK2) | | | | |
| GNB1 | Human | MGC2819 | 2964393 | BC004186 |
| GNG2 | Human | MGC22743 | 4243482 | BC020774 |

### Technical Problem

Despite the existence of a number of non-radioactive assays, there is a continued need for new non-radioactive ligand binding assays for G protein-coupled receptors for drug screening. These assays should be highly specific and provide a clear signal which is readily detectable over background noise. Preferably, these assays should be homogeneous in nature, obviating the requirement for a washing and separation steps and making the assays suitable for compound screening purposes, particularly high throughput drug screening. More preferably, there is a need for additional live cell assays with all the benefits involved in such assays. These problems are resolved by the provision of the assay methods of the present invention, which allow monitoring of activation and deactivation of GPCRs and can be used for testing ligand binding to GPCRs.

### SUMMARY OF THE INVENTION

According to a first aspect of the present invention, there is provided a method for testing for the binding of a ligand to a G Protein-Coupled Receptor (GPCR) in an enzyme complementation assay, the method comprising:
a) providing a fluid sample comprising a GPCR, a Gβ subunit and a Gγ subunit, the Gβ or the Gγ subunit comprising an enzyme fragment which acts as an enzyme donor (ED);
b) adding a G Protein Coupled Receptor Kinase (GRK) or a protein construct comprising the C terminal PH domain thereof to the fluid sample wherein the G Protein Coupled Receptor Kinase (GRK) or the protein construct having G Protein Coupled Receptor Kinase activity comprises an enzyme fragment which acts as an enzyme acceptor (EA) which is capable of enzyme complementation with the enzyme donor (ED);
c) adding a ligand to the fluid sample to allow binding of the ligand to the GPCR to promote association between the GRK or the protein construct and the Gβ subunit and the Gγ subunit and thereby allow enzyme complementation between the enzyme donor (ED) and the enzyme acceptor (EA) to form an active enzyme;
d) adding a substrate of the active enzyme to the fluid sample; and
e) detecting a change in an optical signal resulting from the activity of the active enzyme on the substrate as a measure of ligand binding.

In one aspect, the enzyme fragment is an enzyme acceptor (EA) or enzyme donor (ED) selected from the group of enzymes consisting of β-galactosidase, β-lactamase, dihydrofolate reductase, luciferase, ubiquitinase, alkaline phosphatase and tryptophan synthase.

In another aspect, the enzyme acceptor (EA) is a fragment of β-galactosidase and the enzyme donor (ED) is a fragment of β-galactosidase.

In a further aspect, the GRK is either GRK2 or GRK3. In another aspect, the protein construct is the C-terminal PH Domain of GRK2 or GRK 3.

Preferably, the GRK is GRK2, the enzyme acceptor (EA) is a fragment of β-galactosidase and the enzyme donor (ED) is a fragment of β-galactosidase.

Optionally, the GRK is GRK3, the enzyme acceptor (EA) is a fragment of β-galactosidase and the enzyme donor (ED) is a fragment of β-galactosidase.

Optionally, the protein construct is the C-terminal PH Domain of GRK2 or GRK 3, the enzyme acceptor (EA) is a fragment of β-galactosidase and the enzyme donor (ED) is a fragment of β-galactosidase

In one aspect, the enzyme donor (ED) of β-galactosidase has the sequence disclosed in SEQ ID NO: 1.

In another aspect, the enzyme acceptor (EA) of β-galactosidase has the sequence disclosed in SEQ ID NO: 2.

In a further aspect, the GPCR is in the form of a membrane preparation.

Preferably, the method is an homogeneous assay.

The optical signal is a luminescent signal

In a second aspect of the present invention, there is provided a cell-based assay for testing for the binding of a ligand to a G Protein Coupled Receptor (GPCR) in an enzyme complementation assay, the method comprising:
a) providing a cell expressing a GPCR, a Gβ subunit and a Gγ subunit, the Gβ or the Gγ subunit comprising an enzyme fragment which acts as an enzyme donor (ED);
b) the cell further expressing a G Protein Coupled Receptor Kinase (GRK) or a protein construct comprising the C terminal PH domain thereof wherein the G Protein Coupled Receptor Kinase (GRK) or the protein construct comprises an enzyme fragment which acts as an enzyme acceptor (EA) which is capable of enzyme complementation with the enzyme donor (ED);
c) adding a ligand to the cell to allow binding of the ligand to the GPCR to promote association between the GRK or the protein construct and the Gβ and the Gγ subunits and thereby enzyme complementation between the enzyme donor (ED) and the enzyme acceptor (EA) to form an active enzyme;
d) lysing the cell to provide a cellular lysate;
e) adding a substrate of the active enzyme to the cellular lysate; and
f) detecting a change in an optical signal resulting from the activity of the active enzyme on the substrate as a measure of ligand binding.

In one aspect, the enzyme fragment is an enzyme acceptor (EA) or enzyme donor (ED) selected from the group of enzymes consisting of β-galactosidase, β-lactamase, dihydrofolate reductase, luciferase, ubiquitinase, alkaline phosphatase and tryptophan synthase.

In another aspect, the enzyme acceptor (EA) is a fragment of β-galactosidase and the enzyme donor (ED) is a fragment of β-galactosidase.

In a further aspect, the GRK is either GRK2 or GRK3.

In another aspect, the protein construct is the C-terminal PH Domain of GRK2 or GRK3.

In one aspect, the GRK is GRK2, the enzyme acceptor is a fragment of β-galactosidase and the enzyme donor (ED) is a fragment of β-galactosidase.

In another aspect, the GRK is GRK3, the enzyme acceptor (EA) is a fragment of β-galactosidase and the enzyme donor (ED) is a fragment of β-galactosidase.

In a further aspect, the protein construct s the C-terminal PH Domain of GRK2 or GRK3, the enzyme acceptor (EA) is a fragment of β-galactosidase and the enzyme donor (ED) is a fragment of β-galactosidase.

In one aspect, wherein the enzyme donor (ED) of β-galactosidase has the sequence disclosed in SEQ ID NO: 1.

In another aspect, the enzyme acceptor (EA) of β-galactosidase has the sequence disclosed in SEQ ID NO: 2.

According to a third aspect of the present invention, there is provided a cell expressing
a) a G Protein Coupled Receptor (GPCR);
b) a Gβ subunit and a Gγ subunit, said Gβ or the Gγ subunit comprising an enzyme fragment which acts as an enzyme donor (ED); and
c) a G Protein Coupled Receptor Kinase (GRK) or a protein construct comprising the C terminal PH domain thereof, the GRK or the protein construct comprising an enzyme fragment which acts as an enzyme acceptor (EA) which is capable of enzyme complementation with the enzyme donor (ED);

In one aspect, the enzyme fragment is an enzyme acceptor (EA) or an enzyme donor (ED) selected from the group of enzymes consisting of β-galactosidase, β-lactamase, dihydrofolate reductase, luciferase, ubiquitinase, alkaline phosphatase and tryptophan synthase.

In another aspect, the enzyme acceptor (EA) is a fragment of β-galactosidase and the enzyme donor (ED) is a fragment of β-galactosidase.

In one aspect, the GRK is either GRK2 or GRK3.

In another aspect, the protein construct is the C-terminal PH Domain of GRK2 or GRK3.

In one aspect, the GRK is GRK2, the enzyme acceptor is a fragment of β-galactosidase and the enzyme donor (ED) is a fragment of β-galactosidase.

In another aspect, the GRK is GRK3, the enzyme acceptor is a fragment of β-galactosidase and the enzyme donor (ED) is a fragment of β-galactosidase.

In a further aspect, the protein construct is the C-terminal PH Domain of GRK2 or GRK3, the enzyme acceptor (EA) is a fragment of β-galactosidase and the enzyme donor (ED) is a fragment of β-galactosidase.

In one aspect, the enzyme donor (ED) of β-galactosidase has the sequence disclosed in SEQ ID NO: 1.

In another aspect, the enzyme acceptor (EA) of β-galactosidase has the sequence disclosed in SEQ ID NO: 2.

According to a fourth aspect of the present invention, there is provided a protein sequence disclosed in SEQ ID NO: 1 which is the amino acid sequence of the enzyme donor (ED) of β-galactosidase

According to a fifth aspect of the present invention, there is provided a protein sequence disclosed in SEQ ID NO: 2 which is an amino acid sequence of the enzyme acceptor (EA) of β-galactosidase.

In a sixth aspect of the present invention, there is provided a host cell expressing the protein sequence disclosed in SEQ ID NO: 1.

According to a seventh aspect of the present invention, there is provided a host cell expressing the protein sequence disclosed in SEQ ID NO: 2.

In an eighth aspect of the present invention, there is provided a nucleotide sequence disclosed in SEQ ID No: 3. The nucleotide sequence of SEQ ID NO. 3 encodes the β-galactosidase enzyme donor (ED) of SEQ ID NO: 1

According to a ninth aspect of the present invention, there is provided a nucleotide sequence disclosed in SEQ ID NO: 4. The nucleotide sequence of SEQ ID NO: 4 encodes the β-galactosidase enzyme acceptor (EA) peptide of SEQ ID NO: 2

In a tenth aspect of the present invention, there is provided a vector comprising the nucleotide sequence of SEQ ID NO: 3 or SEQ ID NO: 4.

According to an eleventh aspect of the present invention, there is provided a host cell transformed with the vector as hereinbefore described.

In an eleventh aspect of the present invention, there is provided the use of a host cell as hereinbefore described for drug screening, in particular for high throughput drug screening.

### BRIEF DESCRIPTION OF THE DRAWINGS

FIG. 1 is a schematic representation of a Gβ subunit in which the N-terminus is tagged with an enzyme donor (ED) fragment of β-galactosidase wherein 10 indicates N terminally tagged ED-Gβ subunit.

FIG. 2 is a schematic representation of a Gγ subunit in which the N-terminus is tagged with an enzyme donor (ED) fragment of β-galactosidase wherein 110 indicates N-terminally tagged ED-Gγ subunit.

FIG. 3 is a schematic representation illustrating the interaction of GRK2 with the Gβγ subunit. In the diagram, the GRK2 (200) is seen to comprise the N-terminus (220), which is associated with the C-terminal PH domain (230), and the C-terminus (240) having 22 residues missing from its structure; the Gγ2 N-terminus (250) of the Gβγ subunit has 7 residues missing from its structure while the Gγ2 C-terminal membrane targeting lipid modification site (260) is shown, as is the Gβ1 N-terminus (270).

FIG. 4 discloses SEQ ID NO: 1 which is the amino acid sequence of an enzyme donor (ED) of β-galactosidase.

FIG. 5 discloses SEQ ID NO: 2 which is the amino acid sequence of an enzyme acceptor (EA) of β-galactosidase.

FIG. 6 discloses SEQ ID NO: 3 which is a nucleotide sequence encoding the β-galactosidase enzyme donor (ED) peptide of SEQ ID NO: 1.

FIG. 7 discloses SEQ ID NO: 4 which is a nucleotide sequence encoding the β-galactosidase enzyme acceptor (EA) peptide of SEQ ID NO: 2.

FIG. 8 discloses SEQ ID NO: 5 which is an amino acid sequence of a 47-mer β-galactosidase enzyme donor described by Olson and Eglen (Assay and Drug Development Technologies 2007, 5, 97-105).

FIG. 9 depicts the primary structure of β-galactosidase (NT 1-91)—GNB1 and also discloses the amino acid sequence of this peptide as SEQ ID NO: 6.

FIG. 10 depicts the primary structure of β-galactosidase (NT 1-91)—GNB2 and also discloses the amino acid sequence of this peptide as SEQ ID NO: 7.

FIG. 11 depicts the primary structure of β-galactosidase (NT 1-91)—GNB3 and also discloses the amino acid sequence of this peptide as SEQ ID NO: 8.

FIG. 12 depicts the primary structure of β-galactosidase (NT 1-91)—GNB4 and also discloses the amino acid sequence of this peptide as SEQ ID NO: 9.

FIG. 13 depicts the primary structure of β-galactosidase (NT 1-91)—GNB5a and also discloses the amino acid sequence of this peptide as SEQ ID NO: 10.

FIG. 14 depicts the primary structure of β-galactosidase (NT 1-91)—GNB5b and also discloses the amino acid sequence of this peptide as SEQ ID NO: 11.

FIG. 15 depicts the primary structure of β-galactosidase (NT 1-91)—GNG1 and also discloses the amino acid sequence of this peptide as SEQ ID NO: 12.

FIG. 16 depicts the primary structure of β-galactosidase (NT 1-91)—GNG2 and also discloses the amino acid sequence of this peptide as SEQ ID NO: 13.

FIG. 17 depicts the primary structure of β-galactosidase (NT 1-91)—GNG3 and also discloses the amino acid sequence of this peptide as SEQ ID NO: 14.

FIG. 18 depicts the primary structure of β-galactosidase (NT 1-91)—GNG4 and also discloses the amino acid sequence of this peptide as SEQ ID NO: 15.

FIG. 19 depicts the primary structure of β-galactosidase (NT 1-91)—GNG5 and also discloses the amino acid sequence of this peptide as SEQ ID NO: 16.

FIG. 20 depicts the primary structure of β-galactosidase (NT 1-91)—GNG7 and also discloses the amino acid sequence of this peptide as SEQ ID NO: 17.

FIG. 21 depicts the primary structure of β-galactosidase (NT 1-91)—GNG8 and also discloses the amino acid sequence of this peptide as SEQ ID NO: 18.

FIG. 22 depicts the primary structure of β-galactosidase (NT 1-91)—GNG9 and also discloses the amino acid sequence of this peptide as SEQ ID NO: 19.

FIG. 23 depicts the primary structure of β-galactosidase (NT 1-91)—GNG10 and also discloses the amino acid sequence of this peptide as SEQ ID NO: 20.

FIG. 24 depicts the primary structure of β-galactosidase (NT 1-91)—GNG11 and also discloses the amino acid sequence of this peptide as SEQ ID NO: 21.

FIG. 25 depicts the primary structure of β-galactosidase (NT 1-91)—GNG12 and also discloses the amino acid sequence of this peptide as SEQ ID NO: 22.

FIG. 26 depicts the primary structure of β-galactosidase (NT 1-91)—GNG13 and also discloses the amino acid sequence of this peptide as SEQ ID NO: 23.

FIG. 27 depicts the primary structure of GRK2—β-galactosidase (delta 1-41 CT) and also discloses the amino acid sequence of this peptide as SEQ ID NO: 24.

FIG. 28 depicts the primary structure of GRK2 (CT PH domain)—β-galactosidase (delta 1-41 CT) and also discloses the amino acid sequence of this peptide as SEQ ID NO: 25.

FIG. 29 depicts the primary structure of GRK3—β-galactosidase (delta 1-41 CT) and also discloses the amino acid sequence of this peptide as SEQ ID NO: 26.

FIG. 30 depicts the primary structure of GRK3 (CT PH domain)—β-galactosidase (delta 1-41 CT) and also discloses the amino acid sequence of this peptide as SEQ ID NO: 27.

FIG. 31 is a vector diagram of pCORON1000 and also discloses the nucleotide sequence of the vector as SEQ ID NO: 28

FIG. 32 is a vector diagram of pCORON1000 β-galactosidase (NT 1-91)—GNB1 and also discloses the nucleotide sequence of the vector as SEQ ID NO: 29.

FIG. 33 is a vector diagram of pCORON1000 GRK2 β-galactosidase (delta 1-41 CT) and also discloses the nucleotide sequence of the vector as SEQ ID NO: 30.

FIG. 34a discloses SEQ ID NO: 31 which is the amino acid of a linker peptide which is included in SEQ ID NO: 6-27 and 29-30. The function of this peptide is to act as a flexible link, that connects naturally independent peptides moieties thereby generating a single recombinant chimeric fusion protein. The skilled person will appreciate that other suitable linker peptides could be used to carry out this function”.

FIG. 34b discloses SEQ ID NO: 32 which is the nucleotide sequence encoding the linker peptide of SEQ ID NO: 31.

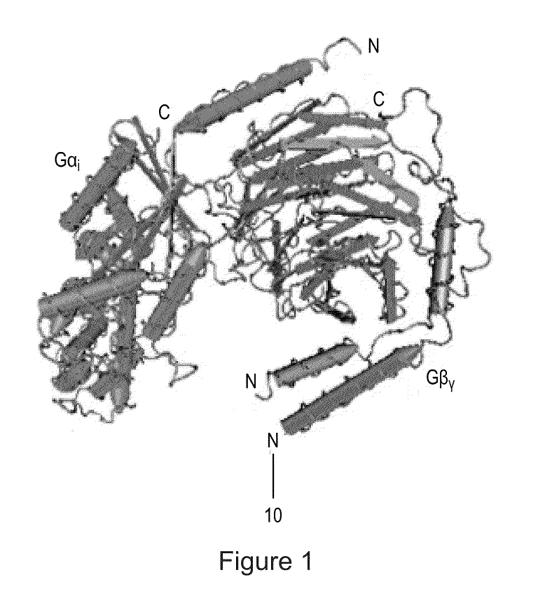

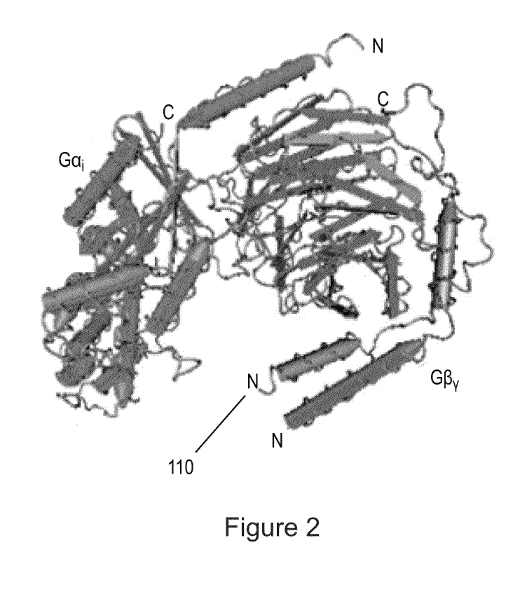

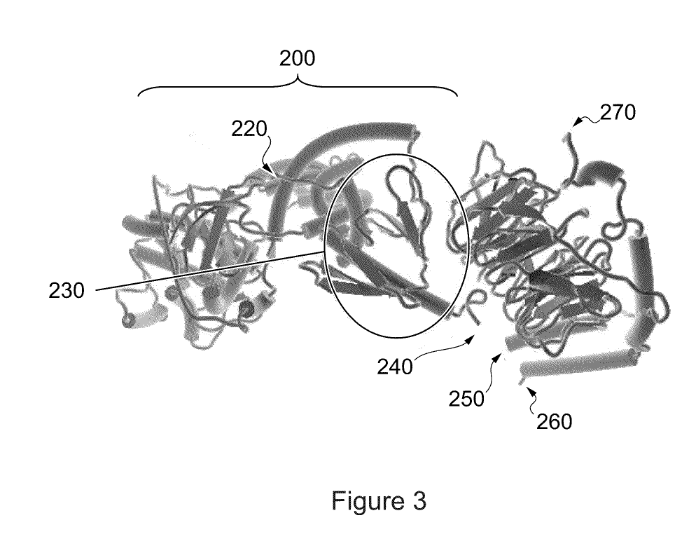

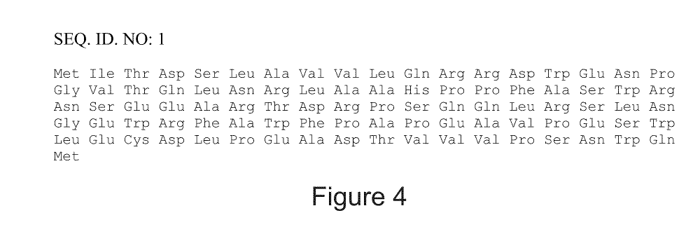

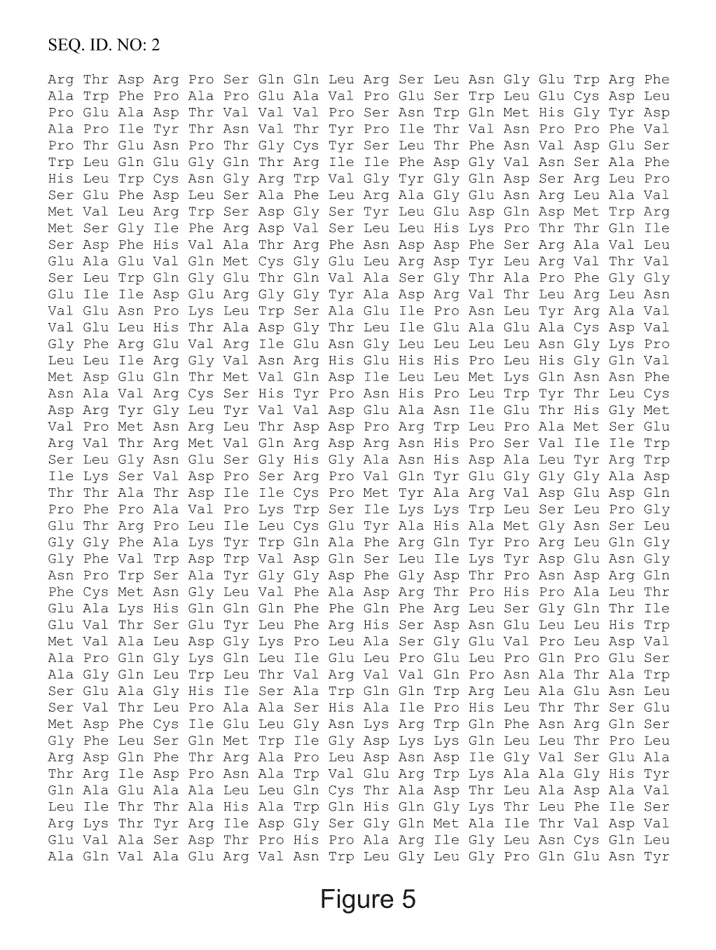

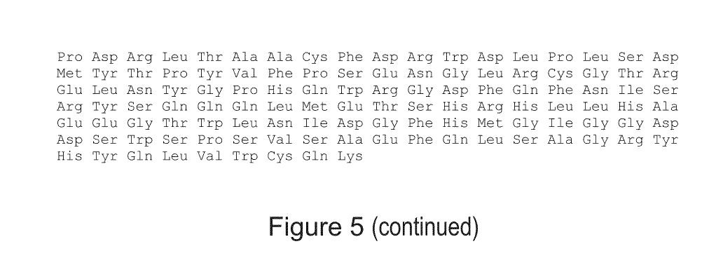

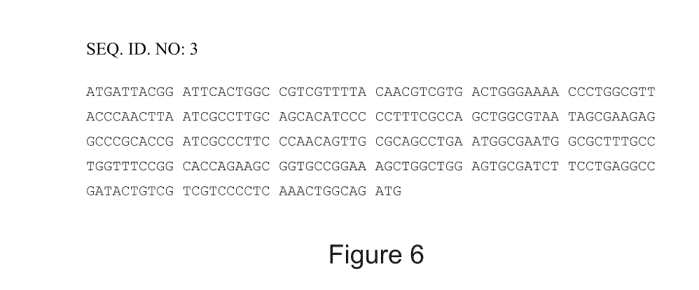

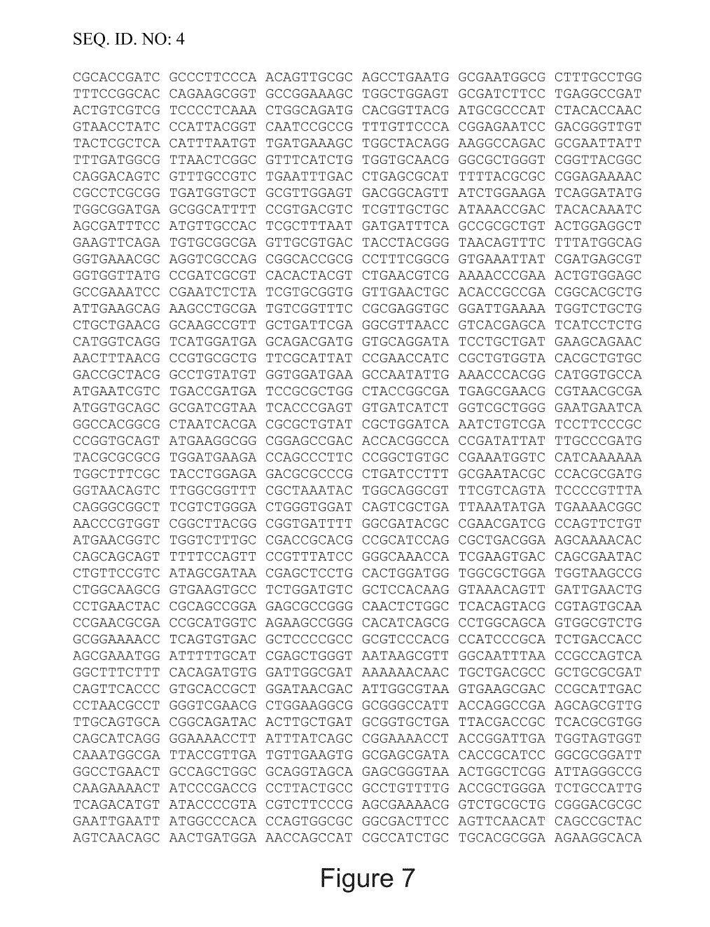

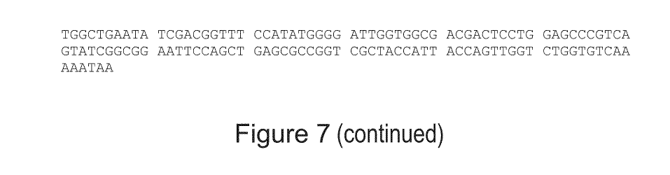

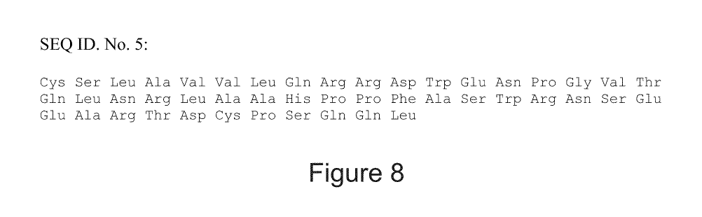

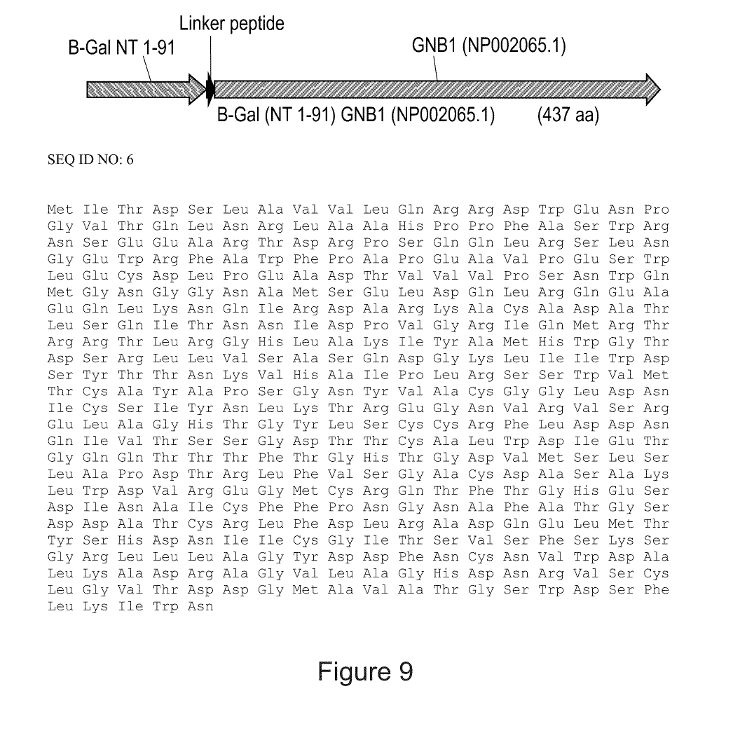

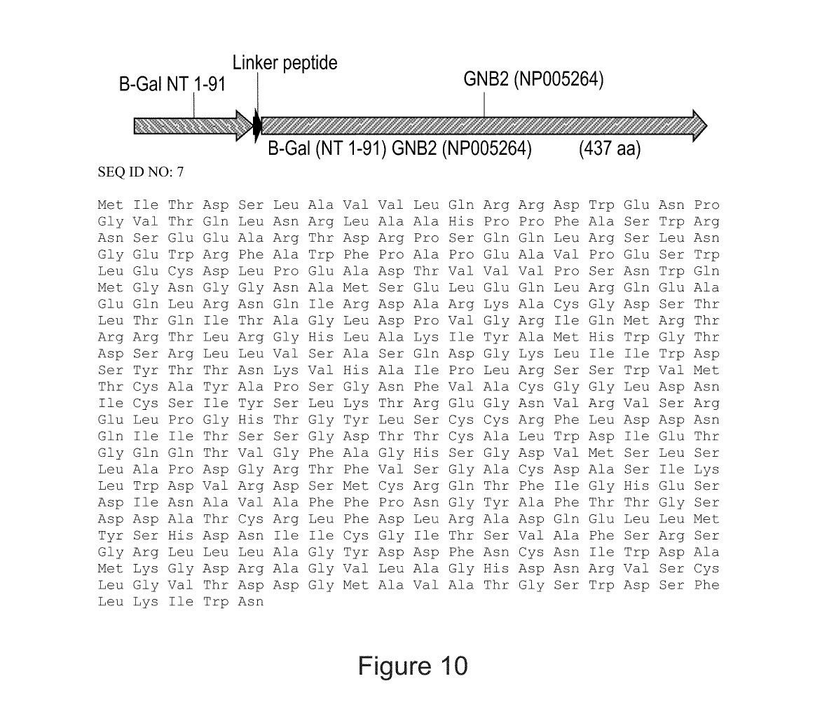

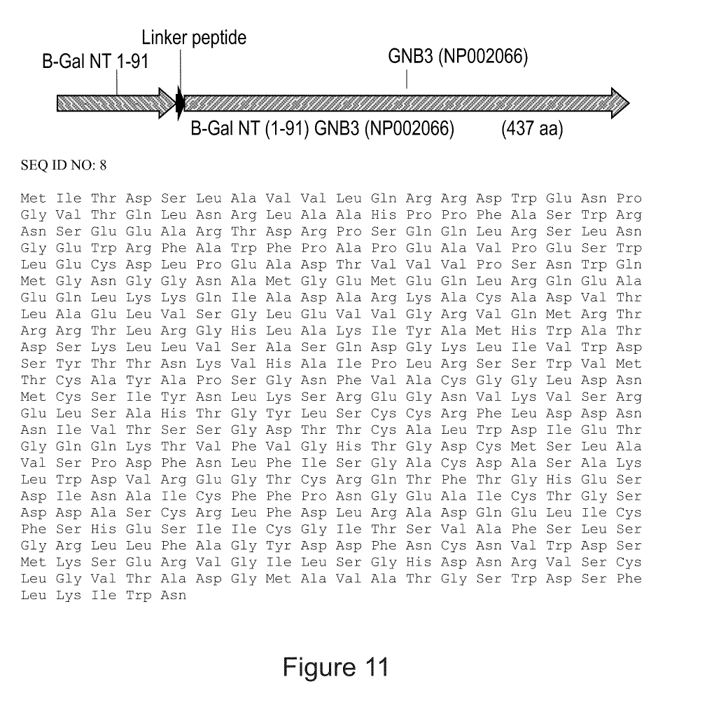

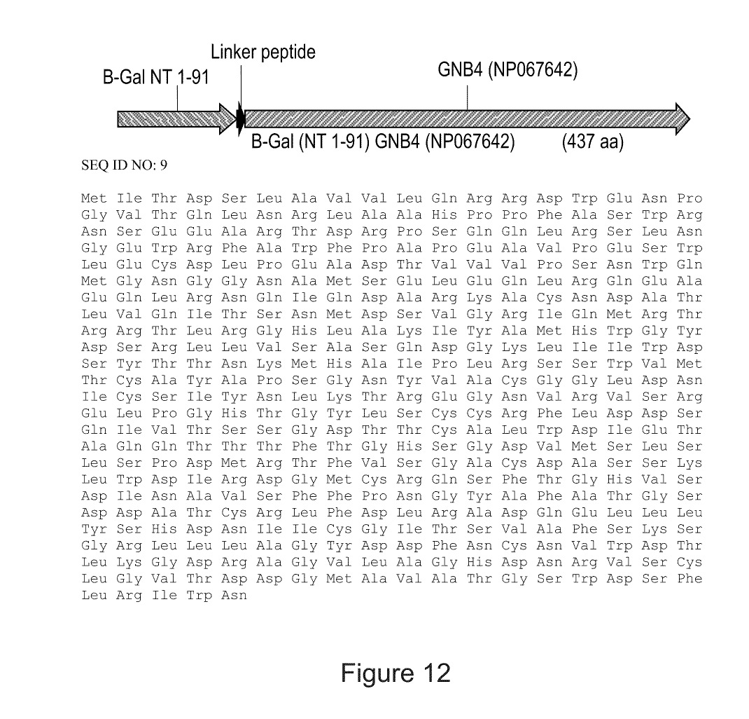

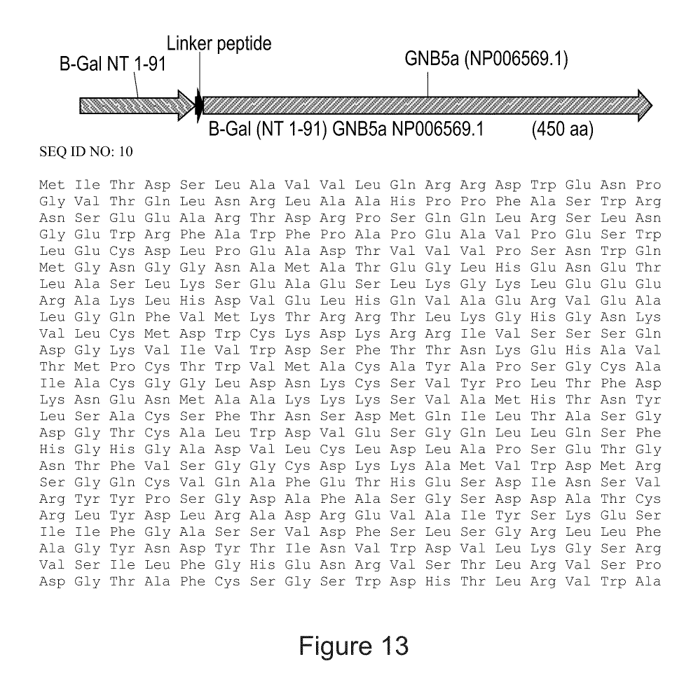

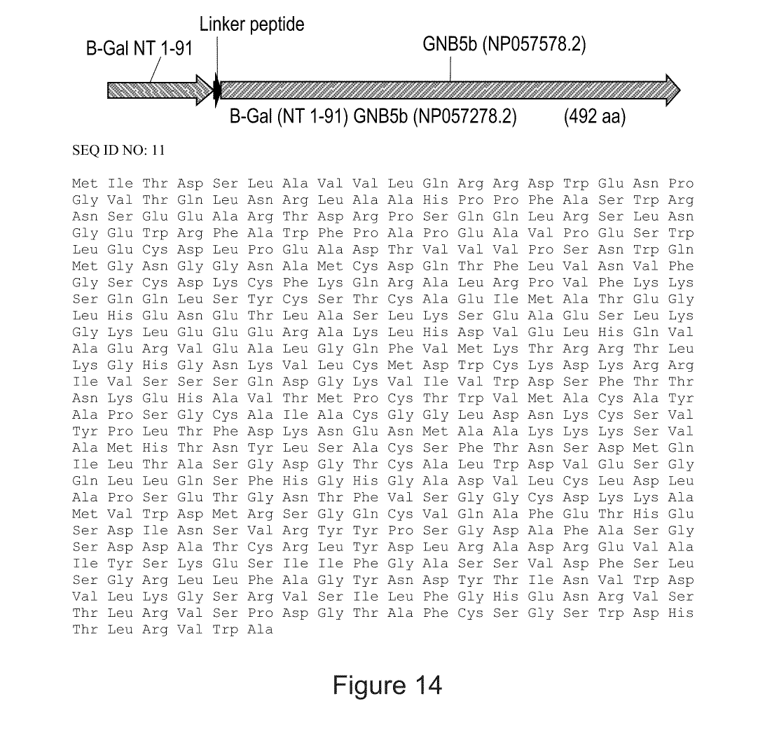

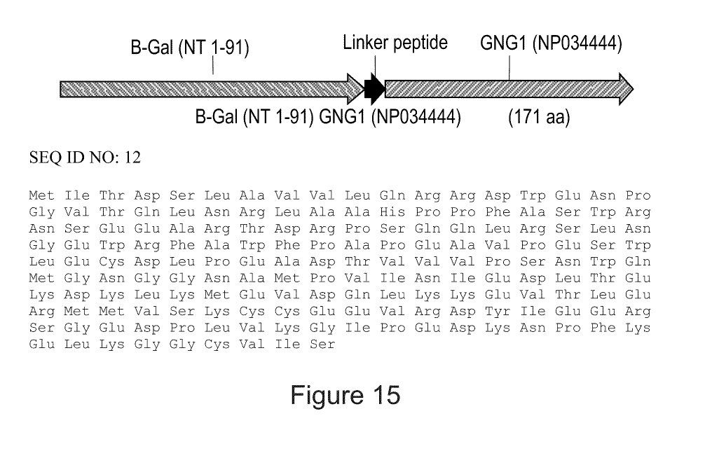

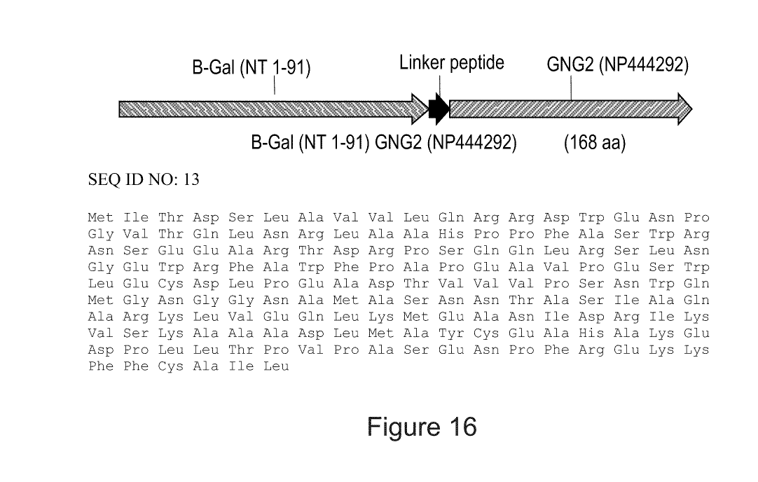

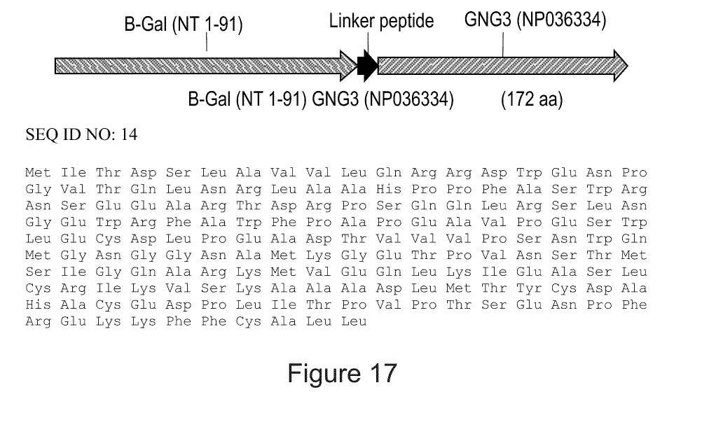

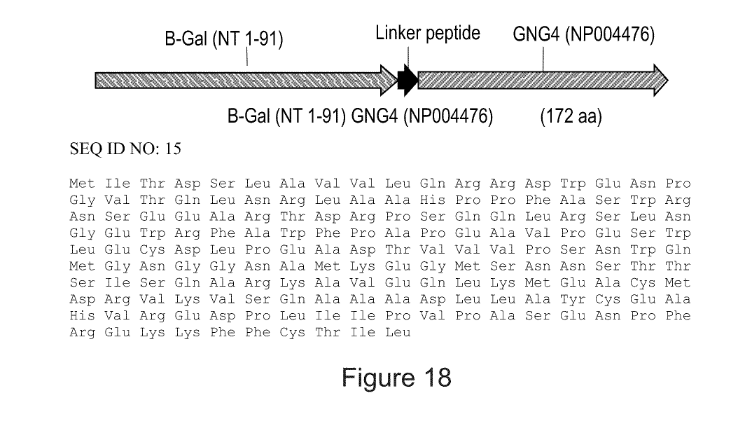

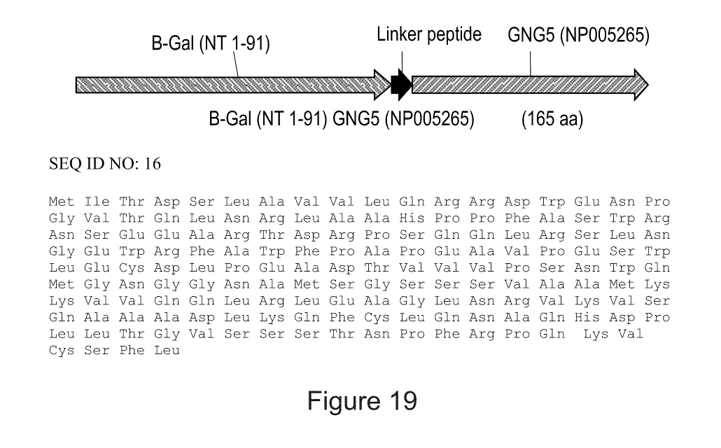

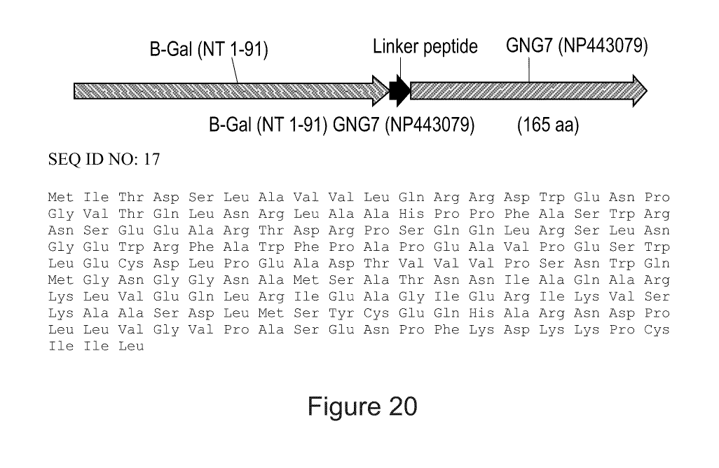

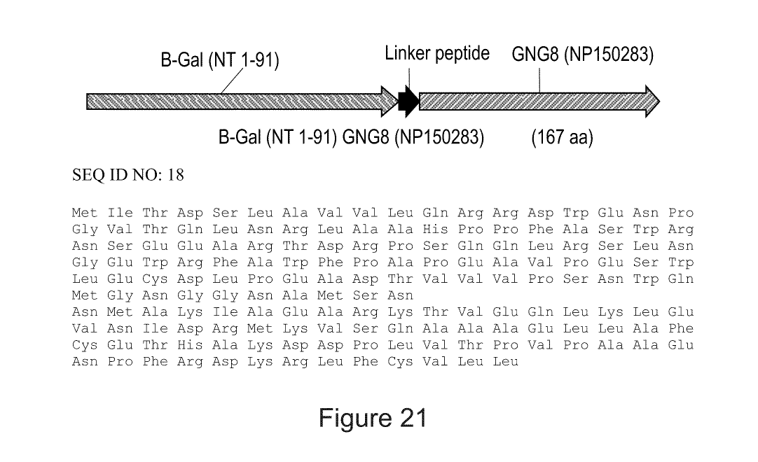

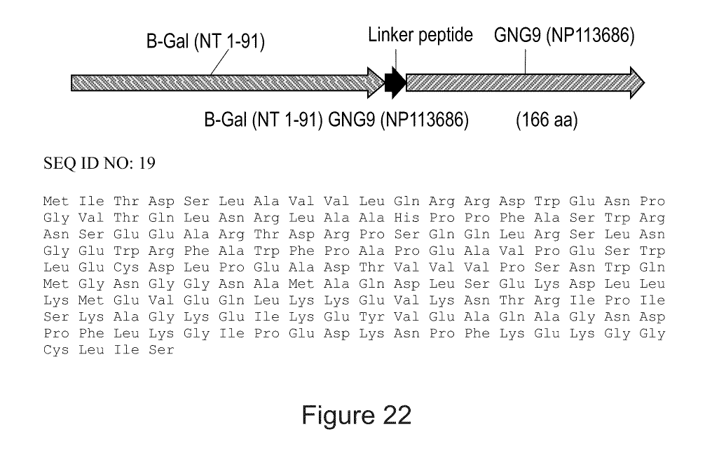

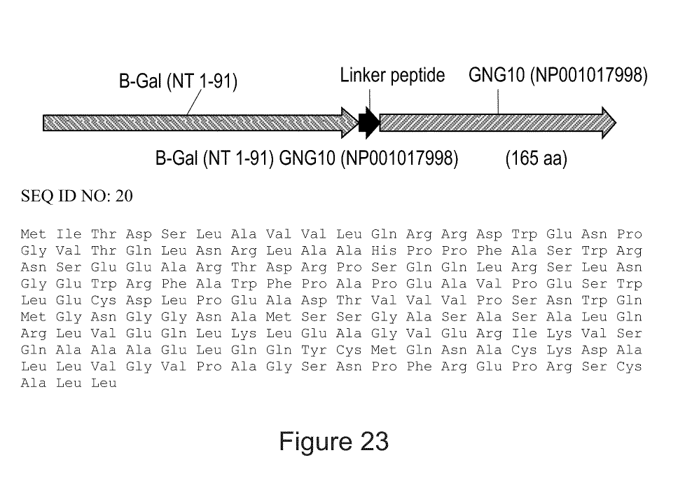

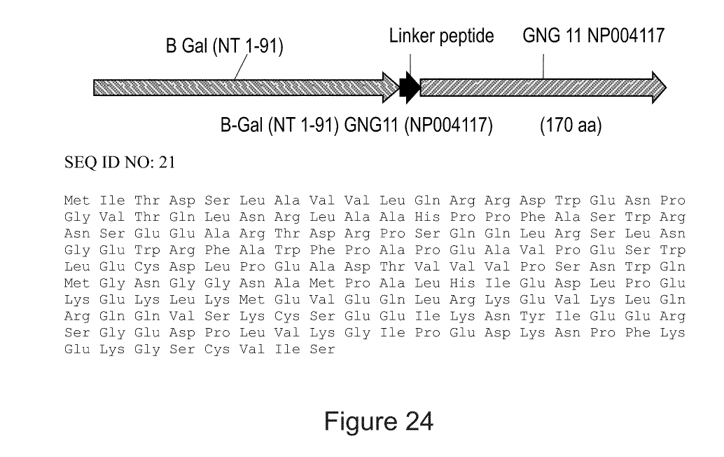

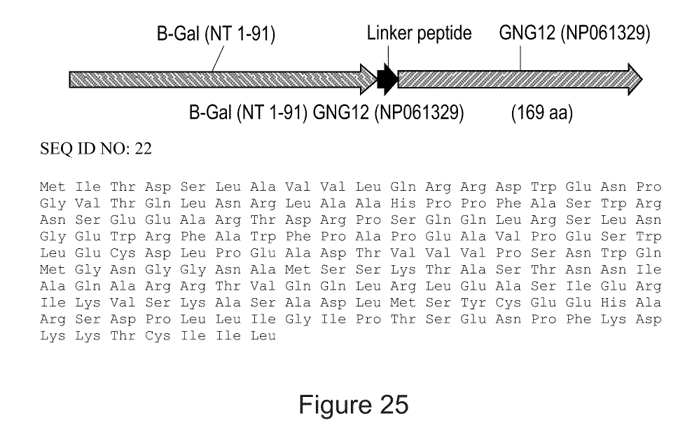

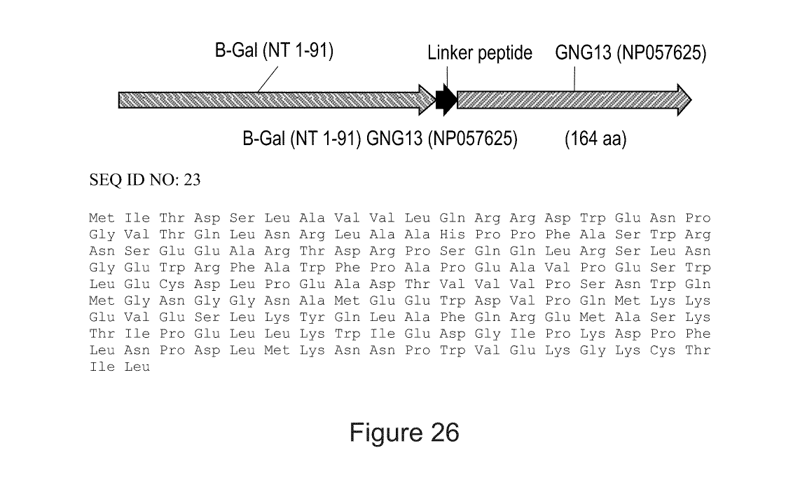

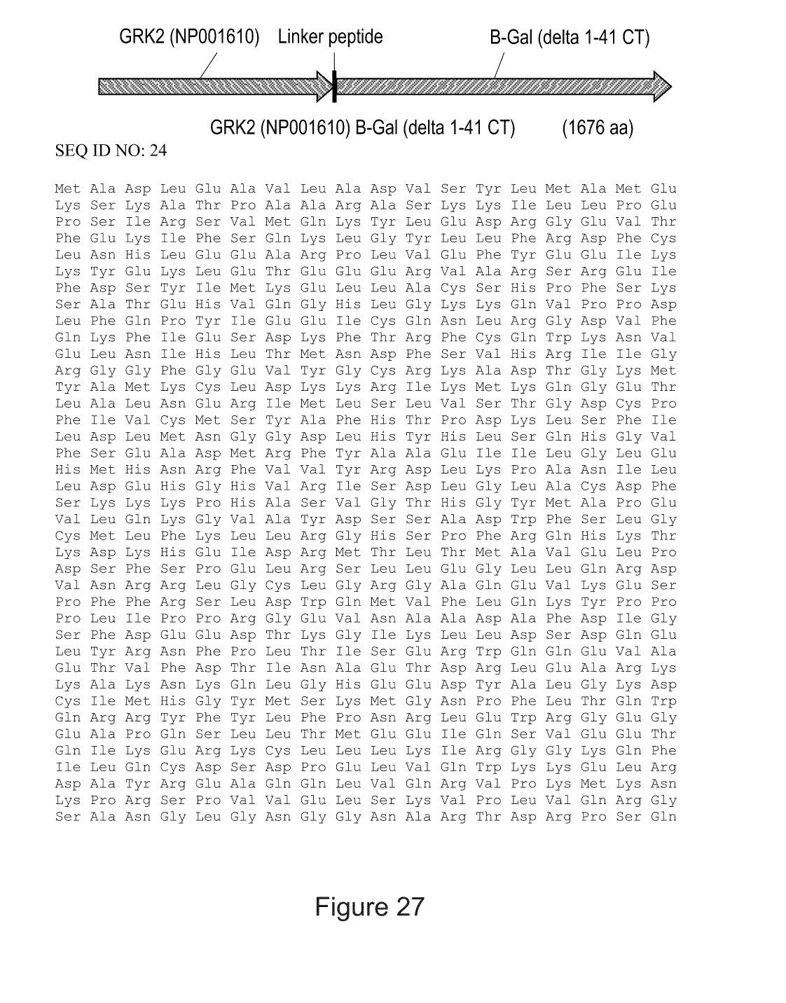

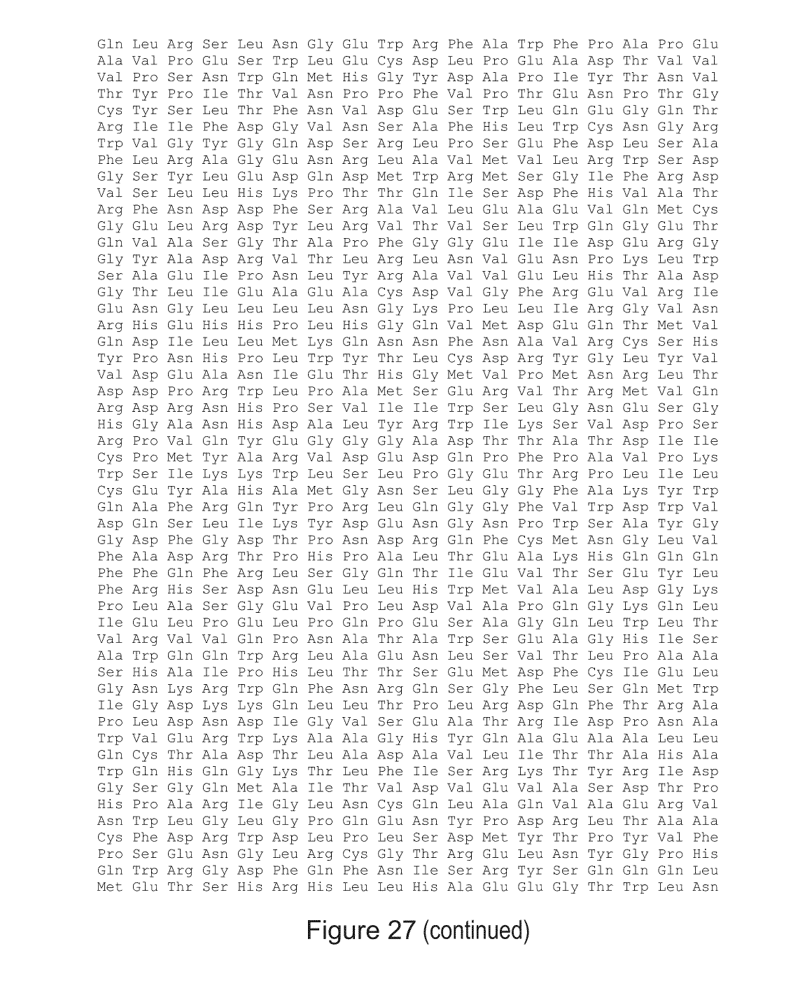

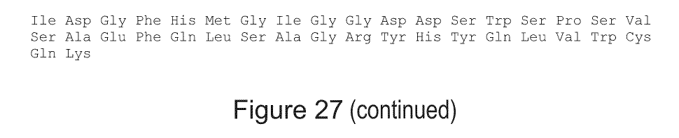

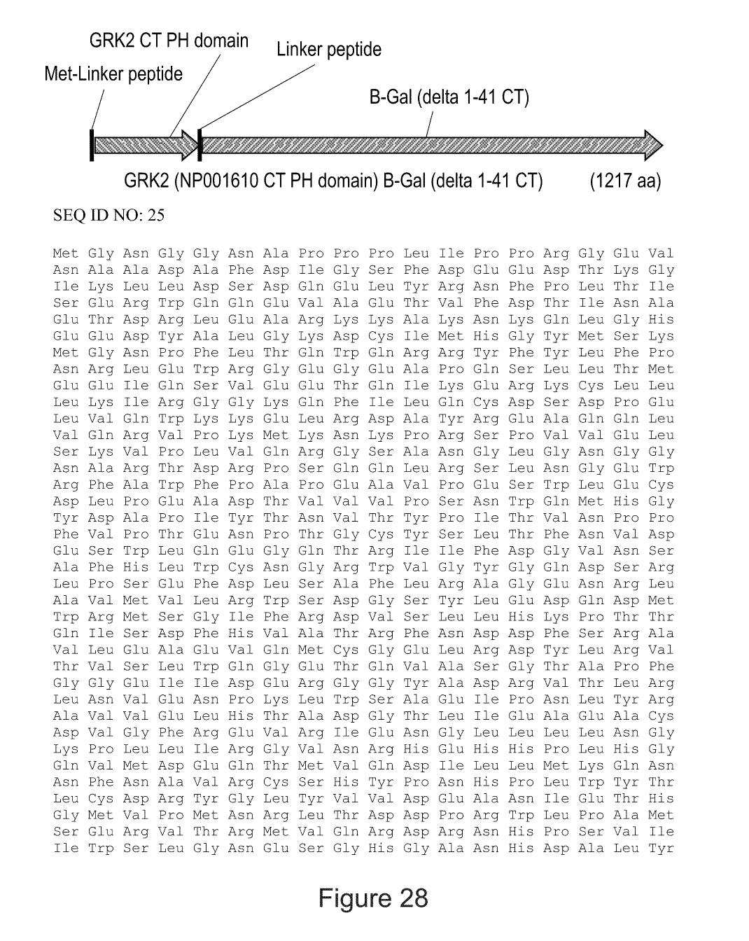

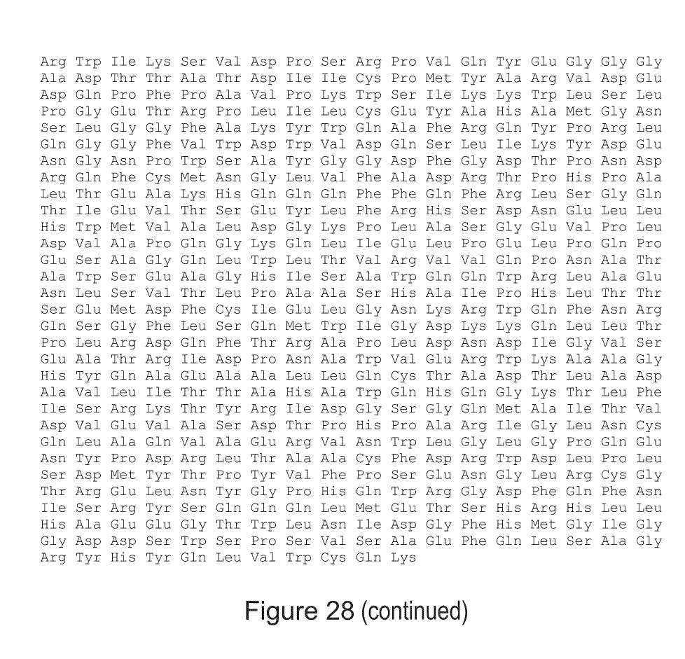

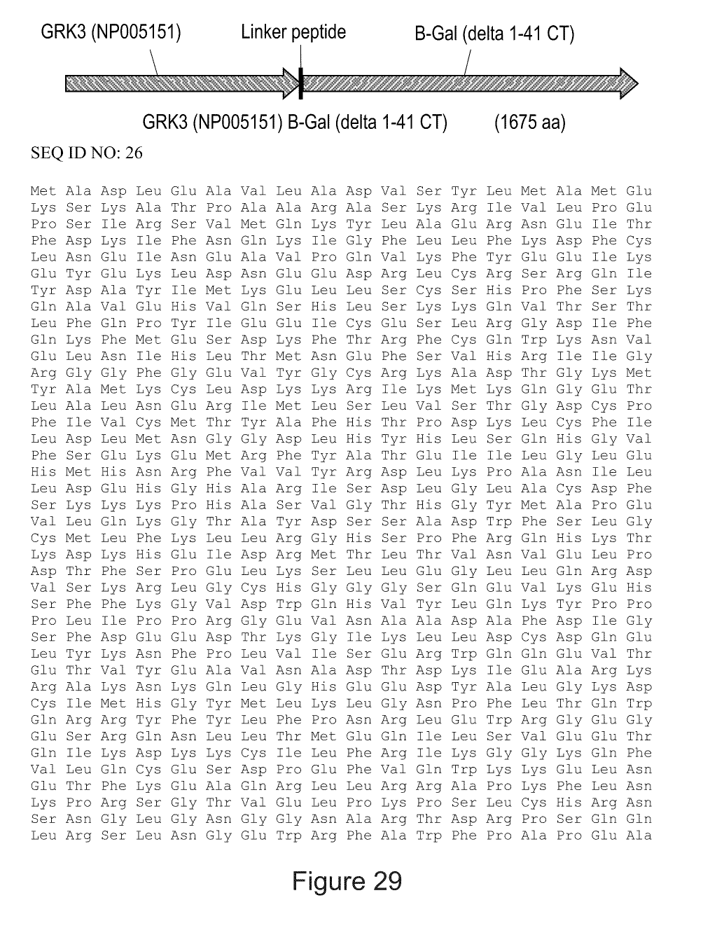

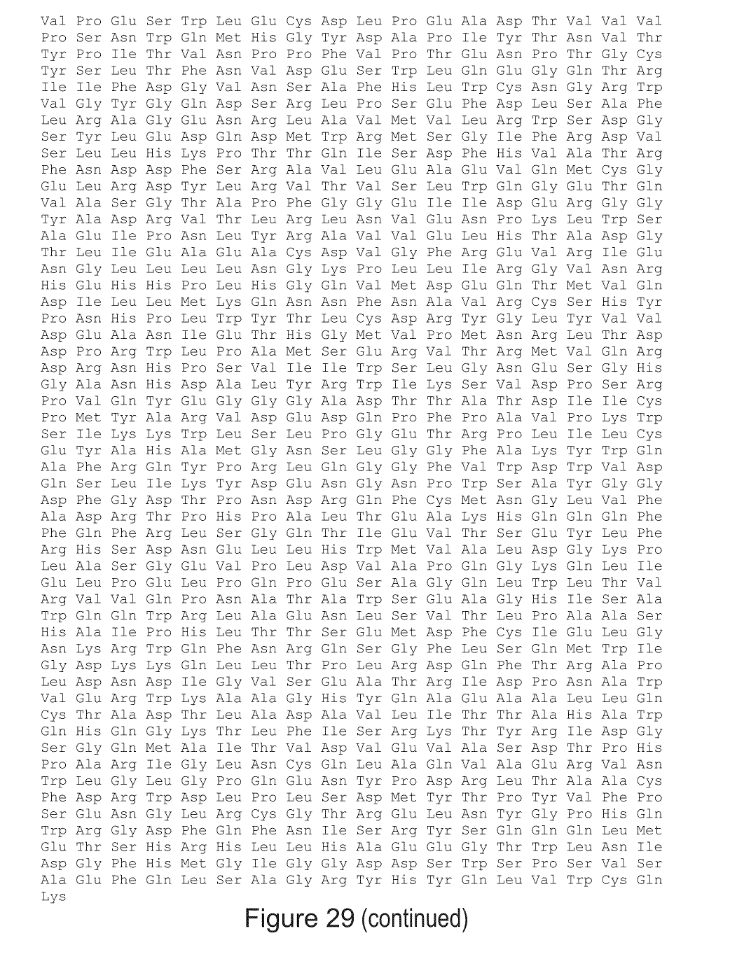

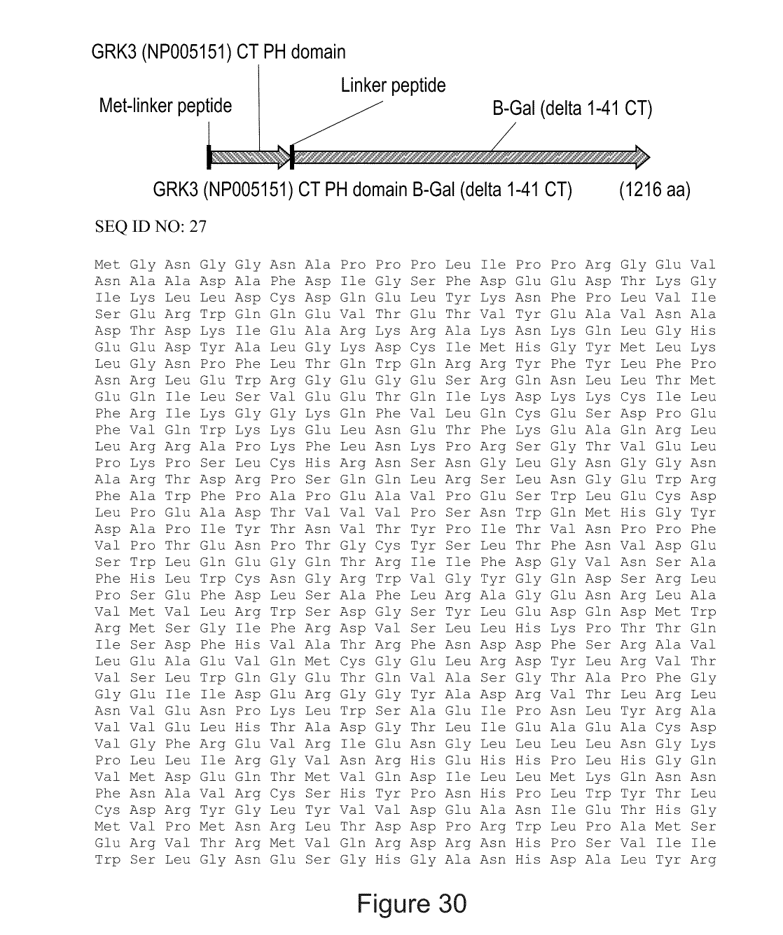

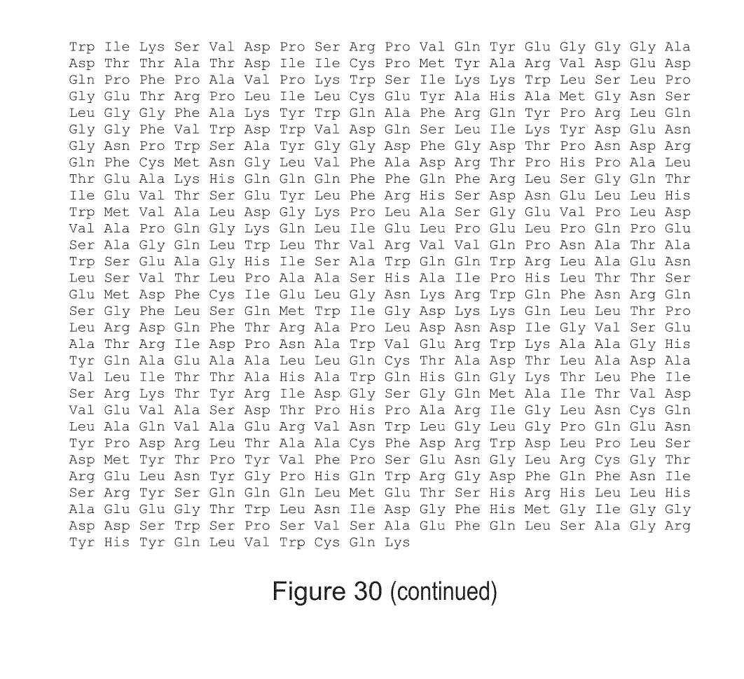

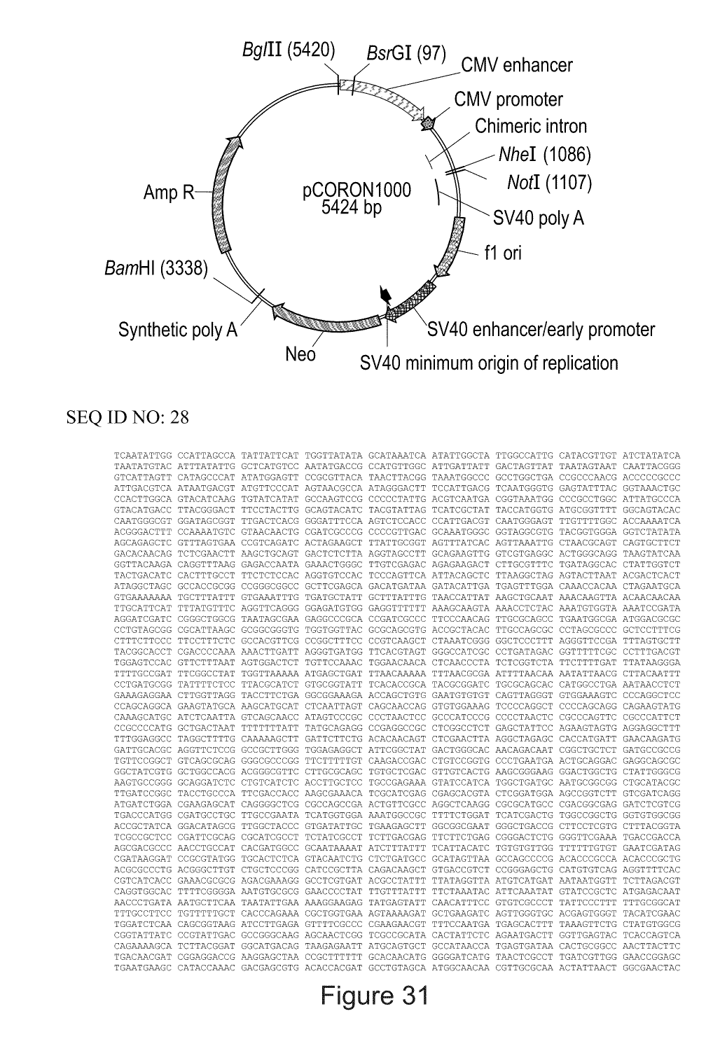

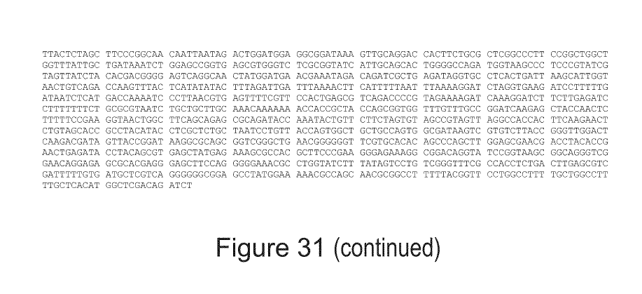

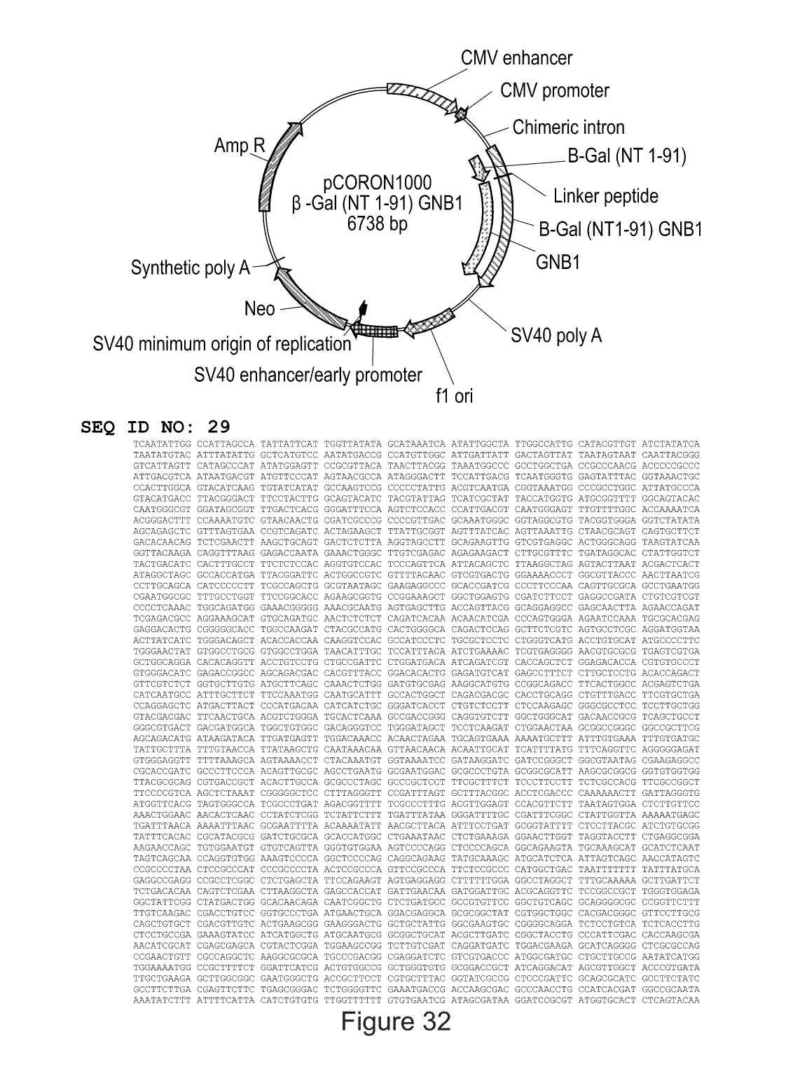

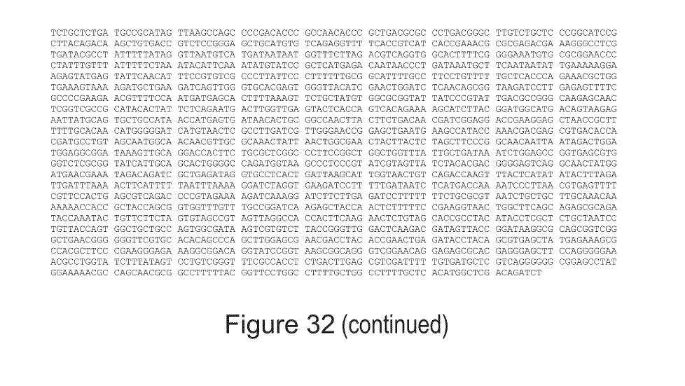

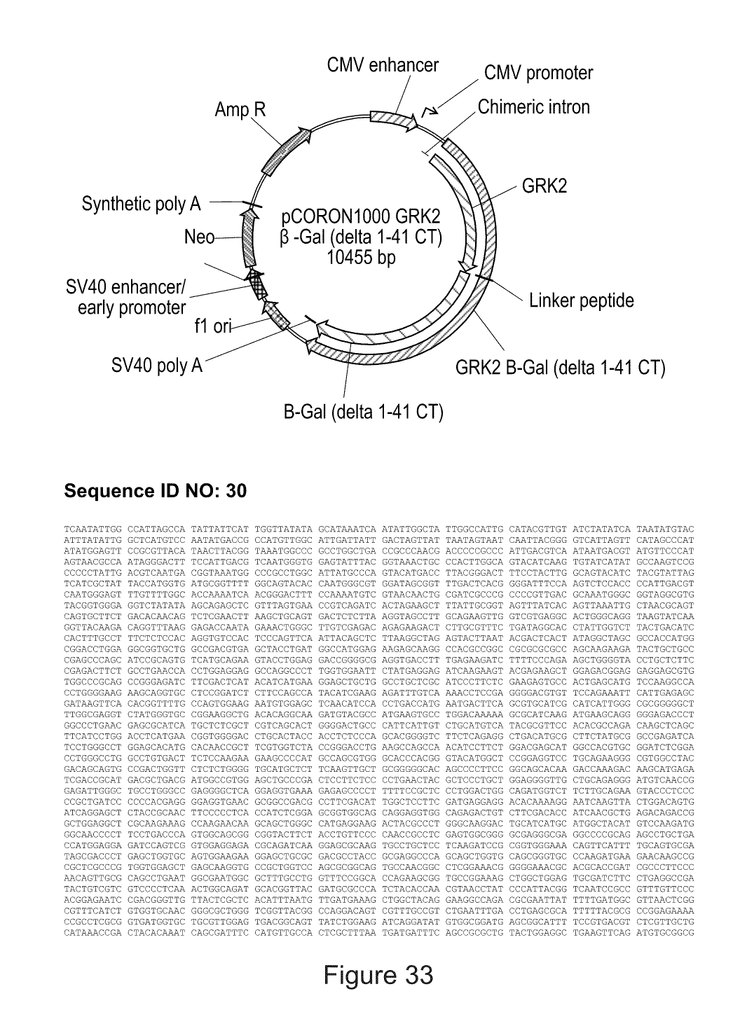

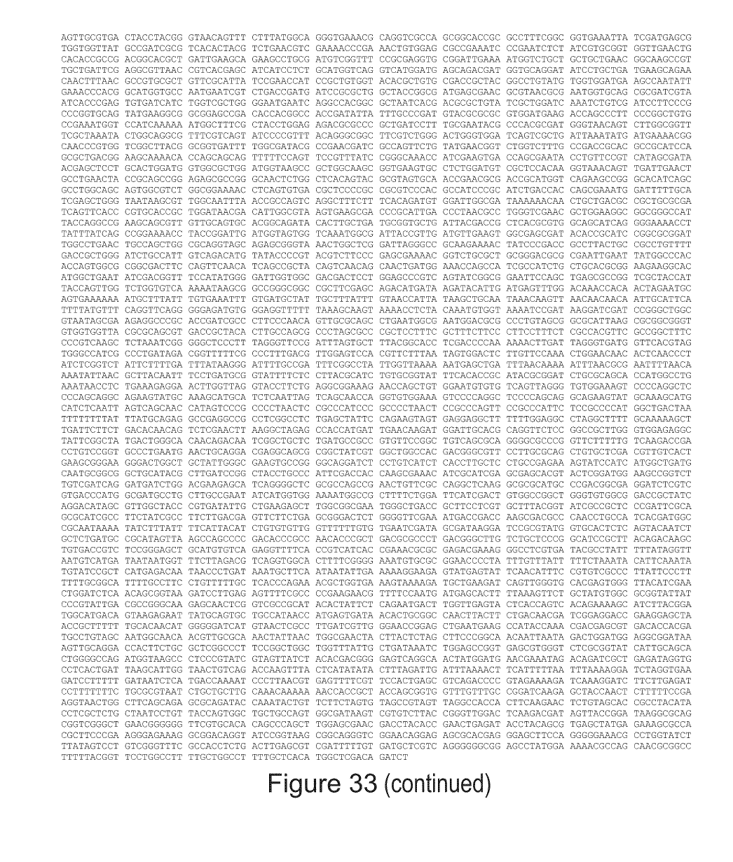

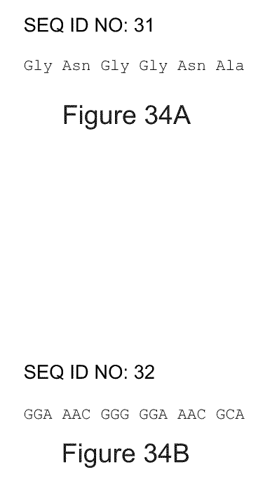

### DETAILED DESCRIPTION OF THE INVENTION

### EXAMPLES

The following examples serve to illustrate embodiments of the present invention. These examples are intended to demonstrate techniques which the present inventors have found to work well in practising the present invention. Hence these examples are detailed so as to provide those of ordinary skill in the art with a complete disclosure and description of the ways in which the methods of this invention may be performed. The following Examples are intended to be exemplary only and changes, modification and alterations can be employed to the conditions described herein, without departing from the scope of the invention.

### 1. Preparation of Genetic Constructs and Transfections of Cells.

### 1.1 Preparation of GRK Enzyme Acceptor Fragments

The method involves creation of a polypeptide chimera comprising a G Protein Coupled Receptor Kinase (GRK) or a protein construct comprising the C terminal PH domain thereof in which the G Protein Coupled Receptor Kinase (GRK) or protein construct comprises an enzyme fragment which acts as β-galactosidase enzyme acceptor (EA) which is capable of enzyme complementation with a β-galactosidase (ED) enzyme donor fragment. The enzyme acceptor component lacks the coding for key amino acids at chosen sites of the β-Gal gene, and the expressed protein would normally exist as an enzymatically inactive dimer.

One of the more widely studied examples of a β-Gal EA peptide is the X90-acceptor peptide that has a deletion in the last 10 amino acids (1013-1023).

The X90 EA peptide exists as a monomer and can be complemented by a corresponding ED fragment of β-Gal, such as CNBr24, a cyanogen bromide digestion product of β-galactosidase consisting of amino acids 990-1023, to reform enzymatically active tetramer (Welphy et al., 1980, Biochem. Biophys. Res. Common., 93, 223).

The GRK chimera protein is constructed, comprising a GRK fused at the C-terminus, to an enzyme acceptor (EA) fragment. In one embodiment, the GRK is GRK2 or GRK3. The cDNA (full length) sequences are available from commercial sources (e.g. Mammalian Gene Collection (MGC), NIH, Maryland, USA).

A vector is constructed (e.g. pCI-neo vector from Promega, Cat no. E1841) using techniques well known coding for the chimera GRK/enzyme acceptor (EA). The pCI-neo Mammalian Expression Vector carries the human cytomegalovirus (CMV) immediate-early enhancer/promoter region to promote constitutive expression of cloned DNA inserts in mammalian cells. This vector also contains the neomycin phosphotransferase gene, a selectable marker for mammalian cells. The pCI-neo Vector can be used for transient expression or for stable expression by selecting transfected cells with the antibiotic G-418.

Transfection of target cells (e.g. mammalian cells) using a transfection agent such as Fugene 6, with the above-described vector is carried out in accordance with Manufacturer's instructions and following the principles outlined by Sambrook and Russell (Molecular Cloning, A Laboratory Manual, 3^{rd} Edition, Volume 3, Chapter 16, Section 16.1-16.54). For example, Fugene 6 and jetPEI, Roche and Polyplus Transfections respectively. In addition transient viral transduction can also be performed using reagents such as adenoviral vectors (Ng P and Graham F L. Methods Mol Med. 2002; 69, 389-414).

The resulting transfected cells are maintained in culture or frozen for later use according to standard practices. These cells express the desired GRK-EA chimera protein, as described above.

### 1.2 Preparation of Gβ and Gγβ-Galactosidase Enzyme Donor Fragments

G⊕ and Gγβ-galactosidase enzyme donor fragments are prepared in a similar manner to that described for the GRK enzyme acceptor fragments above using standard molecular biological techniques according to Sambrook and Russell (Molecular Cloning, A Laboratory Manual).

In one embodiment of the present invention, the β-galactosidase enzyme donor fragment has the amino acid sequence shown in SEQ ID NO: 1. In another embodiment, the β-galactosidase enzyme donor fragment has the amino acid sequence shown in SEQ ID NO: 5:

Cys Ser Leu Ala Val Val Leu Gln Arg Arg Asp Trp
Glu Asn Pro Gly Val Thr Gln Leu Asn Arg Leu Ala
Ala His Pro Pro Phe Ala Ser Trp Arg Asn Ser Glu
Glu Ala Arg Thr Asp Cys Pro Ser Gln Gln Leu.

The 47-mer β-galactosidase enzyme donor being described by Olson and Eglen (Assay and Drug Development Technologies 2007, 5, 97-105).

cDNA (full length) sequences of Gβ and Gγ subunits are available from commercial sources (e.g. Mammalian Gene Collection (MGC), NIH, Maryland, USA).

A vector is constructed (e.g. pCI-neo vector from Promega, Cat no. E1841) using techniques well known in the art coding for the chimera Gβ or Gγ/β-galactosidase enzyme donor fragment. The pCI-neo Mammalian Expression Vector carries the human cytomegalovirus (CMV) immediate-early enhancer/promoter region to promote constitutive expression of cloned DNA inserts in mammalian cells. This vector also contains the neomycin phosphotransferase gene, a selectable marker for mammalian cells. The pCI-neo Vector can be used for transient expression or for stable expression by selecting transfected cells with the antibiotic G-418.

Transfection of target cells (e.g. mammalian cells) using a transfection agent such as Fugene 6, with the above-described vector is carried out in accordance with Manufacturer's instructions and following the principles outlined by Sambrook and Russell (Molecular Cloning, A Laboratory Manual, 3^{rd} Edition, Volume 3, Chapter 16, Section 16.1-16.54). For example Fugene 6 and jetPEI, Roche and Polyplus Transfections respectively. In addition transient viral transduction can also be performed using reagents such as adenoviral vectors (Ng P and Graham F L. Methods Mol Med. 2002; 69, 389-414).

### 1.3 Preparation of Cells Expressing Dual Constructs

Cells expressing both GRK β-galactosidase enzyme acceptor fragments and Gβ and/or Gγβ-galactosidase enzyme donor fragments are prepared by co-transfecting cells with the vectors described in 1.1 and 1.2 above.

The expression vectors described herein allows for the generation of stable cell lines by techniques well known in the art.

### 2. GPCR Assays

Methods of carrying out GPCR assays are well documented in the literature and are well known in the art.

By way of illustration only, and without limitation to the specific assays disclosed, the following techniques are described to demonstrate different assays for utilising the methods of the invention to test for ligand binding to a GPCR.

### 2.1 Assay Methods

Intact cells expressing a GPCR, a Gβ subunit which comprises a β-galactosidase enzyme donor (ED) fragment and a G Protein Coupled Receptor Kinase (GRK) comprising a β-galactosidase enzyme acceptor (EA) are allowed to come into contact in a tube (microwell) in the presence of a suitable buffer. In the presence of a suitable GPCR ligand (e.g. isoproterenol, noradrenaline, salmeterol, denopamine) the GPCR becomes activated, leading to a close proximity of the Gβ-ED and the GRK-EA fragments which will lead to β-galactosidase enzyme complementation. Upon lysis of the cells, with a suitable lysis agent (e.g. detergent, e.g. TRITON® X-100 or TWEEN™ 20) and addition of a suitable β-galactosidase substrate such as the pro-luminescent 1,2-dioxetane substrate (alternative substrates include, for example, 5-acetylaminofluorescein di-b-D-galactopyranoside (X-gal) from Invitrogen; 5-Iodo-3-indolyl-beta -D-galactopyranoside from Sigma; or 5-acetylaminofluorescein di-b-D-galactopyranoside from Invitrogen), an optical signal is generated which can be detected by, for example, a photomultiplier device.

In this system, a signal increase arises from a higher degree of β-galactosidase complementation which is directly proportional to the potency of activation of the ligand.

It will be understood that this method can be adapted to use recombinant proteins in an acellular approach using a cell-free system utilising cell membranes.

### 2.2 Screening Assay Method for GPCR Activation

Cells which express the appropriate combination of constructs described in section 1.3 above are transferred into a 96 (20,000 pre well) or 384 (5,000 cells per well) well culture plate and incubated overnight at 37° C. in a 5% atmosphere of CO₂. An aliquot (e.g. 5 μ) of a suitable test compound or ligand (e.g. isoproterenol, noradrenaline, salmeterol, denopamine) dissolved or suspended in a non-toxic solvent is added to each well and the plate incubated for 1 hour at 37° C. in a 5% atmosphere of CO₂ to allow enzyme complementation to occur. A lysis reagent (such as an appropriate detergent, e.g. TRITON® X-100 or TWEEN™ 20) is added to each well and the plate incubated for 5 minutes. An appropriate luminescent substrate of β-galactosidase (e.g. 5-acetylaminofluorescein di-b-D-galactopyranoside (X-gal) from Invitrogen; 5-Iodo-3-indolyl-beta-D-galactopyranoside from Sigma; or 5-acetylaminofluorescein di-b-D-galactopyranoside from Invitrogen) is added to each well and the plate incubated for 1 to 18 hour (s) at 37° C. in a 5% CO₂ atmosphere. A change in the optical signal (e.g. fluorescence or luminescence) is read using a plate reader or imager (e.g. LEADSEEKER™, GE Healthcare).

While preferred illustrative embodiments of the present invention are described, one skilled in the art will appreciate that the present invention can be practised by other than the described embodiments, which are presented for purposes of illustration only and not by way of limitation. The present invention is limited only by the claims that follow.

1. A method for testing for the binding of a ligand to a G Protein-Coupled Receptor (GPCR) in an enzyme complementation assay, said method comprising:
a) providing a fluid sample comprising a GPCR, a Gβ subunit and a Gγ subunit, said Gβ or said Gγ subunit comprising an enzyme fragment which acts as an enzyme donor (ED);
b) adding a G Protein Coupled Receptor Kinase (GRK) or a protein construct comprising the C terminal PH domain thereof to said fluid sample wherein said G Protein Coupled Receptor Kinase (GRK) or said protein construct having G Protein Coupled Receptor Kinase activity comprises an enzyme fragment which acts as an enzyme acceptor (EA) which is capable of enzyme complementation with said enzyme donor (ED);
c) adding a ligand to the fluid sample to allow binding of said ligand to the GPCR to promote association between the GRK or the protein construct and the Gβ subunit and the Gγ subunit and thereby allow enzyme complementation between the enzyme donor (ED) and said enzyme acceptor (EA) to form an active enzyme;
d) adding a substrate of said active enzyme to the fluid sample; and
e) detecting a change in an optical signal resulting from the activity of the active enzyme on said substrate as a measure of ligand binding.

2. The method of claim 1, wherein said enzyme fragment is an enzyme acceptor (EA) or enzyme donor (ED) selected from the group of enzymes consisting of β-galactosidase, β-lactamase, dihydrofolate reductase, luciferase, ubiquitinase, alkaline phosphatase and tryptophan synthase.

3. The method of claim 1, wherein the enzyme acceptor (EA) is a fragment of β-galactosidase and the enzyme donor (ED) is a fragment of β-galactosidase.

4. The method of claim 1, wherein the GRK is either GRK2 or GRK3.

5. The method of claim 1, wherein the protein construct is the C-terminal PH Domain of GRK2 or GRK 3.

6. The method of claim 4, wherein the GRK is GRK2, the enzyme acceptor (EA) is a fragment of β-galactosidase and the enzyme donor (ED) is a fragment of β-galactosidase.

7. The method of claim 4, wherein the GRK is GRK3, the enzyme acceptor (EA) is a fragment of β-galactosidase and the enzyme donor (ED) is a fragment of β-galactosidase.

8. The method of claim 5, wherein the enzyme acceptor (EA) is a fragment of β-galactosidase and the enzyme donor (ED) is a fragment of β-galactosidase

9. The method of claim 6, wherein the enzyme donor (ED) of β-galactosidase has the sequence disclosed in SEQ ID NO: 1.

10. The method of claim 6, wherein the enzyme acceptor (EA) of β-galactosidase has the sequence disclosed in SEQ ID NO: 2.

11. The method of claim 1, wherein said GPCR is in the form of a membrane preparation.

12. The method of claim 1, wherein said method is an homogeneous assay.

13. (canceled)

14. A cell-based assay for testing for the binding of a ligand to a G Protein Coupled Receptor (GPCR) in an enzyme complementation assay, said method comprising:
a) providing a cell expressing a GPCR, a Gβ subunit and a Gγ subunit, said Gβ or said Gγ subunit comprising an enzyme fragment which acts as an enzyme donor (ED);
b) said cell further expressing a G Protein Coupled Receptor Kinase (GRK) or a protein construct comprising the C terminal PH domain thereof wherein said G Protein Coupled Receptor Kinase (GRK) or said protein construct comprises an enzyme fragment which acts as an enzyme acceptor (EA) which is capable of enzyme complementation with said enzyme donor (ED);
c) adding a ligand to the cell to allow binding of said ligand to the GPCR to promote association between said GRK or the protein construct and the Gβ and the Gγ subunits and thereby enzyme complementation between the enzyme donor (ED) and said enzyme acceptor (EA) to form an active enzyme;
d) lysing the cell to provide a cellular lysate;
e) adding a substrate of said active enzyme to said cellular lysate; and
f) detecting a change in an optical signal resulting from the activity of the active enzyme on said substrate as a measure of ligand binding.

15-23. (canceled)

24. A cell expressing:
a) a G Protein Coupled Receptor (GPCR);
b) a Gβ subunit and a Gγ subunit, said Gβ or said Gγ subunit comprising an enzyme fragment which acts as an enzyme donor (ED); and
c) a G Protein Coupled Receptor Kinase (GRK) or a protein construct comprising the C terminal PH domain thereof, said GRK or said protein construct comprising an enzyme fragment which acts as an enzyme acceptor (EA) which is capable of enzyme complementation with said enzyme donor (ED).

25-42. (canceled)

43. The method of claim 7, wherein the enzyme donor (ED) of β-galactosidase has the sequence disclosed in SEQ ID NO: 1.

44. The method of claim 8, wherein the enzyme donor (ED) of β-galactosidase has the sequence disclosed in SEQ ID NO: 1.

45. The method of claim 7, wherein the enzyme acceptor (EA) of β-galactosidase has the sequence disclosed in SEQ ID NO: 2.

46. The method of claim 8, wherein the enzyme acceptor (EA) of β-galactosidase has the sequence disclosed in SEQ ID NO: 2.

47. The method of claim 9, wherein the enzyme acceptor (EA) of β-galactosidase has the sequence disclosed in SEQ ID NO: 2.


SEQUENCE LISTING (32 sequences)

SEQ ID NO: 1 - protein, 91 aa, Artificial Sequence
  Synthetic peptide
MITDSLAVVLQRRDWENPGVTQLNRLAAHPPFASWRNSEEARTDRPSQQLRSLNGEWRFAWFPAPEAVPESWLECDLPEADTVVVPSNWQM

SEQ ID NO: 2 - protein, 981 aa, Artificial Sequence
  Synthetic peptide
RTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQK

SEQ ID NO: 3 - DNA, 273 bp, Artificial Sequence
  Synthetic oligonucleotide
atgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatg

SEQ ID NO: 4 - DNA, 2946 bp, Artificial Sequence
  Synthetic oligonucleotide
cgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 5 - protein, 47 aa, Artificial Sequence
  Synthetic peptide
CSLAVVLQRRDWENPGVTQLNRLAAHPPFASWRNSEEARTDCPSQQL

SEQ ID NO: 6 - protein, 437 aa, Artificial Sequence
  Synthetic peptide
MITDSLAVVLQRRDWENPGVTQLNRLAAHPPFASWRNSEEARTDRPSQQLRSLNGEWRFAWFPAPEAVPESWLECDLPEADTVVVPSNWQMGNGGNAMSELDQLRQEAEQLKNQIRDARKACADATLSQITNNIDPVGRIQMRTRRTLRGHLAKIYAMHWGTDSRLLVSASQDGKLIIWDSYTTNKVHAIPLRSSWVMTCAYAPSGNYVACGGLDNICSIYNLKTREGNVRVSRELAGHTGYLSCCRFLDDNQIVTSSGDTTCALWDIETGQQTTTFTGHTGDVMSLSLAPDTRLFVSGACDASAKLWDVREGMCRQTFTGHESDINAICFFPNGNAFATGSDDATCRLFDLRADQELMTYSHDNIICGITSVSFSKSGRLLLAGYDDFNCNVWDALKADRAGVLAGHDNRVSCLGVTDDGMAVATGSWDSFLKIWN

SEQ ID NO: 7 - protein, 437 aa, Artificial Sequence
  Synthetic peptide
MITDSLAVVLQRRDWENPGVTQLNRLAAHPPFASWRNSEEARTDRPSQQLRSLNGEWRFAWFPAPEAVPESWLECDLPEADTVVVPSNWQMGNGGNAMSELEQLRQEAEQLRNQIRDARKACGDSTLTQITAGLDPVGRIQMRTRRTLRGHLAKIYAMHWGTDSRLLVSASQDGKLIIWDSYTTNKVHAIPLRSSWVMTCAYAPSGNFVACGGLDNICSIYSLKTREGNVRVSRELPGHTGYLSCCRFLDDNQIITSSGDTTCALWDIETGQQTVGFAGHSGDVMSLSLAPDGRTFVSGACDASIKLWDVRDSMCRQTFIGHESDINAVAFFPNGYAFTTGSDDATCRLFDLRADQELLMYSHDNIICGITSVAFSRSGRLLLAGYDDFNCNIWDAMKGDRAGVLAGHDNRVSCLGVTDDGMAVATGSWDSFLKIWN

SEQ ID NO: 8 - protein, 437 aa, Artificial Sequence [sequence omitted]

SEQ ID NO: 9 - protein, 437 aa, Artificial Sequence
  Synthetic peptide
MITDSLAVVLQRRDWENPGVTQLNRLAAHPPFASWRNSEEARTDRPSQQLRSLNGEWRFAWFPAPEAVPESWLECDLPEADTVVVPSNWQMGNGGNAMSELEQLRQEAEQLRNQIQDARKACNDATLVQITSNMDSVGRIQMRTRRTLRGHLAKIYAMHWGYDSRLLVSASQDGKLIIWDSYTTNKMHAIPLRSSWVMTCAYAPSGNYVACGGLDNICSIYNLKTREGNVRVSRELPGHTGYLSCCRFLDDSQIVTSSGDTTCALWDIETAQQTTTFTGHSGDVMSLSLSPDMRTFVSGACDASSKLWDIRDGMCRQSFTGHVSDINAVSFFPNGYAFATGSDDATCRLFDLRADQELLLYSHDNIICGITSVAFSKSGRLLLAGYDDFNCNVWDTLKGDRAGVLAGHDNRVSCLGVTDDGMAVATGSWDSFLRIWN

SEQ ID NO: 10 - protein, 450 aa, Artificial Sequence
  Synthetic peptide
MITDSLAVVLQRRDWENPGVTQLNRLAAHPPFASWRNSEEARTDRPSQQLRSLNGEWRFAWFPAPEAVPESWLECDLPEADTVVVPSNWQMGNGGNAMATEGLHENETLASLKSEAESLKGKLEEERAKLHDVELHQVAERVEALGQFVMKTRRTLKGHGNKVLCMDWCKDKRRIVSSSQDGKVIVWDSFTTNKEHAVTMPCTWVMACAYAPSGCAIACGGLDNKCSVYPLTFDKNENMAAKKKSVAMHTNYLSACSFTNSDMQILTASGDGTCALWDVESGQLLQSFHGHGADVLCLDLAPSETGNTFVSGGCDKKAMVWDMRSGQCVQAFETHESDINSVRYYPSGDAFASGSDDATCRLYDLRADREVAIYSKESIIFGASSVDFSLSGRLLFAGYNDYTINVWDVLKGSRVSILFGHENRVSTLRVSPDGTAFCSGSWDHTLRVWA

SEQ ID NO: 11 - protein, 492 aa, Artificial Sequence
  Synthetic peptide
MITDSLAVVLQRRDWENPGVTQLNRLAAHPPFASWRNSEEARTDRPSQQLRSLNGEWRFAWFPAPEAVPESWLECDLPEADTVVVPSNWQMGNGGNAMCDQTFLVNVFGSCDKCFKQRALRPVFKKSQQLSYCSTCAEIMATEGLHENETLASLKSEAESLKGKLEEERAKLHDVELHQVAERVEALGQFVMKTRRTLKGHGNKVLCMDWCKDKRRIVSSSQDGKVIVWDSFTTNKEHAVTMPCTWVMACAYAPSGCAIACGGLDNKCSVYPLTFDKNENMAAKKKSVAMHTNYLSACSFTNSDMQILTASGDGTCALWDVESGQLLQSFHGHGADVLCLDLAPSETGNTFVSGGCDKKAMVWDMRSGQCVQAFETHESDINSVRYYPSGDAFASGSDDATCRLYDLRADREVAIYSKESIIFGASSVDFSLSGRLLFAGYNDYTINVWDVLKGSRVSILFGHENRVSTLRVSPDGTAFCSGSWDHTLRVWA

SEQ ID NO: 12 - protein, 171 aa, Artificial Sequence
  Synthetic peptide
MITDSLAVVLQRRDWENPGVTQLNRLAAHPPFASWRNSEEARTDRPSQQLRSLNGEWRFAWFPAPEAVPESWLECDLPEADTVVVPSNWQMGNGGNAMPVINIEDLTEKDKLKMEVDQLKKEVTLERMMVSKCCEEVRDYIEERSGEDPLVKGIPEDKNPFKELKGGCVIS

SEQ ID NO: 13 - protein, 168 aa, Artificial Sequence
  Synthetic peptide
MITDSLAVVLQRRDWENPGVTQLNRLAAHPPFASWRNSEEARTDRPSQQLRSLNGEWRFAWFPAPEAVPESWLECDLPEADTVVVPSNWQMGNGGNAMASNNTASIAQARKLVEQLKMEANIDRIKVSKAAADLMAYCEAHAKEDPLLTPVPASENPFREKKFFCAIL

SEQ ID NO: 14 - protein, 172 aa, Artificial Sequence
  Synthetic peptide
MITDSLAVVLQRRDWENPGVTQLNRLAAHPPFASWRNSEEARTDRPSQQLRSLNGEWRFAWFPAPEAVPESWLECDLPEADTVVVPSNWQMGNGGNAMKGETPVNSTMSIGQARKMVEQLKIEASLCRIKVSKAAADLMTYCDAHACEDPLITPVPTSENPFREKKFFCALL

SEQ ID NO: 15 - protein, 172 aa, Artificial Sequence
  Synthetic peptide
MITDSLAVVLQRRDWENPGVTQLNRLAAHPPFASWRNSEEARTDRPSQQLRSLNGEWRFAWFPAPEAVPESWLECDLPEADTVVVPSNWQMGNGGNAMKEGMSNNSTTSISQARKAVEQLKMEACMDRVKVSQAAADLLAYCEAHVREDPLIIPVPASENPFREKKFFCTIL

SEQ ID NO: 16 - protein, 165 aa, Artificial Sequence
  Synthetic peptide
MITDSLAVVLQRRDWENPGVTQLNRLAAHPPFASWRNSEEARTDRPSQQLRSLNGEWRFAWFPAPEAVPESWLECDLPEADTVVVPSNWQMGNGGNAMSGSSSVAAMKKVVQQLRLEAGLNRVKVSQAAADLKQFCLQNAQHDPLLTGVSSSTNPFRPQKVCSFL

SEQ ID NO: 17 - protein, 165 aa, Artificial Sequence
  Synthetic peptide
MITDSLAVVLQRRDWENPGVTQLNRLAAHPPFASWRNSEEARTDRPSQQLRSLNGEWRFAWFPAPEAVPESWLECDLPEADTVVVPSNWQMGNGGNAMSATNNIAQARKLVEQLRIEAGIERIKVSKAASDLMSYCEQHARNDPLLVGVPASENPFKDKKPCIIL

SEQ ID NO: 18 - protein, 167 aa, Artificial Sequence
  Synthetic peptide
MITDSLAVVLQRRDWENPGVTQLNRLAAHPPFASWRNSEEARTDRPSQQLRSLNGEWRFAWFPAPEAVPESWLECDLPEADTVVVPSNWQMGNGGNAMSNNMAKIAEARKTVEQLKLEVNIDRMKVSQAAAELLAFCETHAKDDPLVTPVPAAENPFRDKRLFCVLL

SEQ ID NO: 19 - protein, 166 aa, Artificial Sequence
  Synthetic peptide
MITDSLAVVLQRRDWENPGVTQLNRLAAHPPFASWRNSEEARTDRPSQQLRSLNGEWRFAWFPAPEAVPESWLECDLPEADTVVVPSNWQMGNGGNAMAQDLSEKDLLKMEVEQLKKEVKNTRIPISKAGKEIKEYVEAQAGNDPFLKGIPEDKNPFKEKGGCLIS

SEQ ID NO: 20 - protein, 165 aa, Artificial Sequence
  Synthetic peptide
MITDSLAVVLQRRDWENPGVTQLNRLAAHPPFASWRNSEEARTDRPSQQLRSLNGEWRFAWFPAPEAVPESWLECDLPEADTVVVPSNWQMGNGGNAMSSGASASALQRLVEQLKLEAGVERIKVSQAAAELQQYCMQNACKDALLVGVPAGSNPFREPRSCALL

SEQ ID NO: 21 - protein, 170 aa, Artificial Sequence
  Synthetic peptide
MITDSLAVVLQRRDWENPGVTQLNRLAAHPPFASWRNSEEARTDRPSQQLRSLNGEWRFAWFPAPEAVPESWLECDLPEADTVVVPSNWQMGNGGNAMPALHIEDLPEKEKLKMEVEQLRKEVKLQRQQVSKCSEEIKNYIEERSGEDPLVKGIPEDKNPFKEKGSCVIS

SEQ ID NO: 22 - protein, 169 aa, Artificial Sequence
  Synthetic peptide
MITDSLAVVLQRRDWENPGVTQLNRLAAHPPFASWRNSEEARTDRPSQQLRSLNGEWRFAWFPAPEAVPESWLECDLPEADTVVVPSNWQMGNGGNAMSSKTASTNNIAQARRTVQQLRLEASIERIKVSKASADLMSYCEEHARSDPLLIGIPTSENPFKDKKTCIIL

SEQ ID NO: 23 - protein, 164 aa, Artificial Sequence
  Synthetic peptide
MITDSLAVVLQRRDWENPGVTQLNRLAAHPPFASWRNSEEARTDRPSQQLRSLNGEWRFAWFPAPEAVPESWLECDLPEADTVVVPSNWQMGNGGNAMEEWDVPQMKKEVESLKYQLAFQREMASKTIPELLKWIEDGIPKDPFLNPDLMKNNPWVEKGKCTIL

SEQ ID NO: 24 - protein, 1676 aa, Artificial Sequence
  Synthetic peptide
MADLEAVLADVSYLMAMEKSKATPAARASKKILLPEPSIRSVMQKYLEDRGEVTFEKIFSQKLGYLLFRDFCLNHLEEARPLVEFYEEIKKYEKLETEEERVARSREIFDSYIMKELLACSHPFSKSATEHVQGHLGKKQVPPDLFQPYIEEICQNLRGDVFQKFIESDKFTRFCQWKNVELNIHLTMNDFSVHRIIGRGGFGEVYGCRKADTGKMYAMKCLDKKRIKMKQGETLALNERIMLSLVSTGDCPFIVCMSYAFHTPDKLSFILDLMNGGDLHYHLSQHGVFSEADMRFYAAEIILGLEHMHNRFVVYRDLKPANILLDEHGHVRISDLGLACDFSKKKPHASVGTHGYMAPEVLQKGVAYDSSADWFSLGCMLFKLLRGHSPFRQHKTKDKHEIDRMTLTMAVELPDSFSPELRSLLEGLLQRDVNRRLGCLGRGAQEVKESPFFRSLDWQMVFLQKYPPPLIPPRGEVNAADAFDIGSFDEEDTKGIKLLDSDQELYRNFPLTISERWQQEVAETVFDTINAETDRLEARKKAKNKQLGHEEDYALGKDCIMHGYMSKMGNPFLTQWQRRYFYLFPNRLEWRGEGEAPQSLLTMEEIQSVEETQIKERKCLLLKIRGGKQFILQCDSDPELVQWKKELRDAYREAQQLVQRVPKMKNKPRSPVVELSKVPLVQRGSANGLGNGGNARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQK

SEQ ID NO: 25 - protein, 1217 aa, Artificial Sequence
  Synthetic peptide
MGNGGNAPPPLIPPRGEVNAADAFDIGSFDEEDTKGIKLLDSDQELYRNFPLTISERWQQEVAETVFDTINAETDRLEARKKAKNKQLGHEEDYALGKDCIMHGYMSKMGNPFLTQWQRRYFYLFPNRLEWRGEGEAPQSLLTMEEIQSVEETQIKERKCLLLKIRGGKQFILQCDSDPELVQWKKELRDAYREAQQLVQRVPKMKNKPRSPVVELSKVPLVQRGSANGLGNGGNARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQK

SEQ ID NO: 26 - protein, 1675 aa, Artificial Sequence [sequence omitted]

SEQ ID NO: 27 - protein, 1216 aa, Artificial Sequence
  Synthetic peptide
MGNGGNAPPPLIPPRGEVNAADAFDIGSFDEEDTKGIKLLDCDQELYKNFPLVISERWQQEVTETVYEAVNADTDKIEARKRAKNKQLGHEEDYALGKDCIMHGYMLKLGNPFLTQWQRRYFYLFPNRLEWRGEGESRQNLLTMEQILSVEETQIKDKKCILFRIKGGKQFVLQCESDPEFVQWKKELNETFKEAQRLLRRAPKFLNKPRSGTVELPKPSLCHRNSNGLGNGGNARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQK

SEQ ID NO: 28 - DNA, 5424 bp, Artificial Sequence
  Synthetic oligonucleotide
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgccaccgcggccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 29 - DNA, 6738 bp, Artificial Sequence
  Synthetic oligonucleotide
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgccaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgggaaacgggggaaacgcaatgagtgagcttgaccagttacggcaggaggccgagcaacttaagaaccagattcgagacgccaggaaagcatgtgcagatgcaactctctctcagatcacaaacaacatcgacccagtgggaagaatccaaatgcgcacgaggaggacactgcgggggcacctggccaagatctacgccatgcactggggcacagactccaggcttctcgtcagtgcctcgcaggatggtaaacttatcatctgggacagctacaccaccaacaaggtccacgccatccctctgcgctcctcctgggtcatgacctgtgcatatgccccttctgggaactatgtggcctgcggtggcctggataacatttgctccatttacaatctgaaaactcgtgaggggaacgtgcgcgtgagtcgtgagctggcaggacacacaggttacctgtcctgctgccgattcctggatgacaatcagatcgtcaccagctctggagacaccacgtgtgccctgtgggacatcgagaccggccagcagacgaccacgtttaccggacacactggagatgtcatgagcctttctcttgctcctgacaccagactgttcgtctctggtgcttgtgatgcttcagccaaactctgggatgtgcgagaaggcatgtgccggcagaccttcactggccacgagtctgacatcaatgccatttgcttctttccaaatggcaatgcatttgccactggctcagacgacgccacctgcaggctgtttgaccttcgtgctgaccaggagctcatgacttactcccatgacaacatcatctgcgggatcacctctgtctccttctccaagagcgggcgcctcctccttgctgggtacgacgacttcaactgcaacgtctgggatgcactcaaagccgaccgggcaggtgtcttggctgggcatgacaaccgcgtcagctgcctgggcgtgactgacgatggcatggctgtggcgacagggtcctgggatagcttcctcaagatctggaactaagcggccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 30 - DNA, 10455 bp, Artificial Sequence
  Synthetic peptide
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagtgactctcttaaggtagccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcgccaccatggcggacctggaggcggtgctggccgacgtgagctacctgatggccatggagaagagcaaggccacgccggccgcgcgcgccagcaagaagatactgctgcccgagcccagcatccgcagtgtcatgcagaagtacctggaggaccggggcgaggtgacctttgagaagatcttttcccagaagctggggtacctgctcttccgagacttctgcctgaaccacctggaggaggccaggcccttggtggaattctatgaggagatcaagaagtacgagaagctggagacggaggaggagcgtgtggcccgcagccgggagatcttcgactcatacatcatgaaggagctgctggcctgctcgcatcccttctcgaagagtgccactgagcatgtccaaggccacctggggaagaagcaggtgcctccggatctcttccagccatacatcgaagagatttgtcaaaacctccgaggggacgtgttccagaaattcattgagagcgataagttcacacggttttgccagtggaagaatgtggagctcaacatccacctgaccatgaatgacttcagcgtgcatcgcatcattgggcgcgggggctttggcgaggtctatgggtgccggaaggctgacacaggcaagatgtacgccatgaagtgcctggacaaaaagcgcatcaagatgaagcagggggagaccctggccctgaacgagcgcatcatgctctcgctcgtcagcactggggactgcccattcattgtctgcatgtcatacgcgttccacacgccagacaagctcagcttcatcctggacctcatgaacggtggggacctgcactaccacctctcccagcacggggtcttctcagaggctgacatgcgcttctatgcggccgagatcatcctgggcctggagcacatgcacaaccgcttcgtggtctaccgggacctgaagccagccaacatccttctggacgagcatggccacgtgcggatctcggacctgggcctggcctgtgacttctccaagaagaagccccatgccagcgtgggcacccacgggtacatggctccggaggtcctgcagaagggcgtggcctacgacagcagtgccgactggttctctctggggtgcatgctcttcaagttgctgcgggggcacagccccttccggcagcacaagaccaaagacaagcatgagatcgaccgcatgacgctgacgatggccgtggagctgcccgactccttctcccctgaactacgctccctgctggaggggttgctgcagagggatgtcaaccggagattgggctgcctgggccgaggggctcaggaggtgaaagagagcccctttttccgctccctggactggcagatggtcttcttgcagaagtaccctcccccgctgatccccccacgaggggaggtgaacgcggccgacgccttcgacattggctccttcgatgaggaggacacaaaaggaatcaagttactggacagtgatcaggagctctaccgcaacttccccctcaccatctcggagcggtggcagcaggaggtggcagagactgtcttcgacaccatcaacgctgagacagaccggctggaggctcgcaagaaagccaagaacaagcagctgggccatgaggaagactacgccctgggcaaggactgcatcatgcatggctacatgtccaagatgggcaaccccttcctgacccagtggcagcggcggtacttctacctgttccccaaccgcctcgagtggcggggcgagggcgaggccccgcagagcctgctgaccatggaggagatccagtcggtggaggagacgcagatcaaggagcgcaagtgcctgctcctcaagatccgcggtgggaaacagttcattttgcagtgcgatagcgaccctgagctggtgcagtggaagaaggagctgcgcgacgcctaccgcgaggcccagcagctggtgcagcgggtgcccaagatgaagaacaagccgcgctcgcccgtggtggagctgagcaaggtgccgctggtccagcgcggcagtgccaacggcctcggaaacgggggaaacgcacgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgttggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataagcggccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatacgcggatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaaggctagagccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgatggccgcaataaaatatctttattttcattacatctgtgtgttggttttttgtgtgaatcgatagcgataaggatccgcgtatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 31 - protein, 6 aa, Artificial Sequence
  Synthetic peptide
GNGGNA

SEQ ID NO: 32 - DNA, 18 bp, Artificial Sequence
  Synthetic oligonucleotide
ggaaacgggggaaacgca